SEQUENCE LISTING (5 sequences)

SEQ ID NO: 1 - protein, 17 aa, Borrelia burgdorferi
MKKYLLGIGLILALIAC

SEQ ID NO: 2 - DNA, 51 bp, Borrelia burgdorferi
atgaaaaaatatttattgggaataggtctaatattagccttaatagcatgt

SEQ ID NO: 3 - protein, 17 aa, Borrelia burgdorferi
MKKYLLGIGLILALIAD

SEQ ID NO: 4 - DNA, 36 bp, Borrelia burgdorferi
ttgggaataggtctaatattagccttaatagcagat

SEQ ID NO: 5 - DNA, 822 bp, Artificial Sequence
  Description of Artificial Sequence Synthetic nucleotide construct
atgaaaaaatatttattgggaataggtctaatattagccttaatagcatgtaagcaaaatgttagcagccttgacgagaaaaacagcgtttcagtagatttgcctggtgaaatgaaagttcttgtaagcaaagaaaaaaacaaagacggcaagtacgatctaattgcaacagtagacaagcttgagcttaaaggaacttctgataaaaacaatggatctggagtacttgaaggcgtaaaagctgacaaaagtaaagtaaaattaacaatttctgacgatctaggtcaaaccacacttgaagttttcaaagaagatggcaaaacactagtatcaaaaaaagtaacttccaaagacaagtcatcaacagaagaaaaattcaatgaaaaaggtgaagtatctgaaaaaataataacaagagcagacggaaccagacttgaatacacaggaattaaaagcgatggatctggaaaagctaaagaggttttaaaaggctttactcttgaaggaaaagtagctaatgataaagtaacattggaagtaaaagaaggaaccgttactttaagtaagaatatttcaaaatctggggaagtttcagttgaacttaatgacactgacagtagtgctgctactaaaaaaactgcagcttggaattcaaaaacttctactttaacaattagtgttaacagcaaaaaaactacacaacttgtgtttactaaacaagacacaataactgtacaaaaatacgactccgcaggtaccaatttagaaggcacagcagtcgaaattaaaacacttgatgaacttaaaaacgctttaaaataa

(19) United States
(12) Patent Application Publication
(10) Pub. No.: US 2009/0324638 A1
(43) Pub. Date: Dec. 31, 2009

(54) LIVE BACTERIAL VACCINE

(75) Inventors: Raymond J. Dattwyler, East Setauket, NY (US); Maria J.C. Gomes-Solecki, Memphis, TN (US); Jozef F.M.L. Seegers, Leiden (NL)

Correspondence Address: VENABLE LLP, P.O. BOX 34385, WASHINGTON, DC, 20043-9998, US

(73) Assignees: Biopeptides Corp., New York, NY (US); Lactrys Octrooi BV, Delft (NL)

(21) Appl. No.: 12/304,634

(22) PCT Filed: Jun. 12, 2006
(86) PCT No.: PCT/US2007/013753

§ 371 Date: Mar. 31, 2009

Related U.S. Application Data
(60) Provisional application No. 60/812,595, filed on Jun. 12, 2006.

(51) Int. Cl. A61K 39/02; C12N 1/21; A61P 31/04

(52) U.S. Cl. 424/200.1; 435/252.3

(57) ABSTRACT

The present invention relates, e.g., to a Lactobacillus bacterium, which (1) expresses a recombinant polypeptide containing a lipoprotein signal sequence from the OspA protein of Borrelia burgdorferi, or an active variant of the leader sequence, operably linked to one or more heterologous polypeptide(s) of interest and/or (2) which comprises an expressible polynucleotide encoding a recombinant polypeptide, wherein the polynucleotide encodes a lipoprotein signal from the OspA protein of Borrelia burgdorferi, or an active variant thereof, which is operably linked to one or more heterologous polypeptide(s) of interest. In one embodiment, the heterologous polypeptide is from Yersinia pestis, the etiologic agent of plague. In another embodiment, the heterologous polypeptide is from Borrelia burgdorferi, the etiologic agent of Lyme disease. Also described are immunogenic compositions, such as live bacterial vaccines, comprising the bacterium; methods for eliciting an immune response against the polypeptide using the bacterium; and kits comprising the bacterium.

This application claims the benefit of the filing date of U.S. provisional application 60/812,595, filed Jun. 12, 2006, which is incorporated by reference herein in its entirety.

This research was supported by U.S. government grants (NIH grant numbers RO1 AI 411582 and 1R44 AI58364-03). The government thus has certain rights in the invention.

### FIELD OF THE INVENTION

The present invention relates, e.g., to a bacterium, such as a Lactobacillus bacterium, which has been recombinantly engineered to express one or more immunogenic polypeptides. The bacterium may be an immunogenic composition, such as a live bacterial vaccine.

### BACKGROUND INFORMATION

Yersinia pestis (Y. pestis), the etiologic agent of plague, is a prime candidate to be used as a bioweapon, for example during a terrorist attack. Whether weaponized or not, this pathogen has attributes that make it an ideal choice to produce mass casualties. An attack with a Y. pestis would undoubtedly be delivered as an aerosol. Pneumonic plague is extremely contagious, highly lethal and there are naturally occurring multiple antibiotic resistant strains.

Only 100 to 500 organisms need to be inhaled to produce pneumonic plague. Post exposure, pneumonia develops within two to three days and it is virtually 100 percent lethal if treatment with efficacious antibiotics is not begun immediately. In addition, as pneumonia develops the victim becomes an excellent vehicle, rapidly spreading the disease. Coughing produces infected droplets and anyone within a radius of two meters from an individual with pneumonic plague has a high risk of becoming infected themselves. Thus, once pneumonic plague develops in a population, unless it is identified and dealt with promptly and effectively, the number of cases will increase exponentially. An attack with an antibiotic resistant strain would vastly complicate our response. One solution is to develop a safe effective vaccine, particularly an oral vaccine, preferably one that can protect against both bubonic and pneumonic plague. Ideally any plague vaccine should produce both a systemic and a mucosal immune response. Currently, there is no plague vaccine licensed in the United States.

The present application describes, e.g., a recombinant bacterial vaccine that can be used against Yersinia pestis, e.g. as an oral vaccine. The method for making and using the bacterium can serve as a platform for the development of a variety of vaccines against other pathogens, including other pathogenic bacteria, viruses, fungi or parasites.

### BRIEF DESCRIPTION OF THE DRAWINGS

FIG. 1 shows Western blot analysis of Lactobacillus plantarum strain 256 (Lp256) expressing variants of B. burgdorferi OspA: LpA, LpAα, and LpAc17d. The size of the wild type OspA, shown in relation to markers of 28 and 35 Kd, is about 31 Kd. FIG. 1A shows immunoblotting to mAb 184.1; FIG. 1B shows immunoblotting to mAb LA2.2; and FIG. 1C shows immunoblotting to mAb 336.1. See Example II for a further discussion of this experiment.

FIG. 2 shows immunoblot detection of LcrV expression. 1: +control (LcrV expressed in E. coli), 2: marker, 3: L. plantarum, no plasmid, 4: L. plantarum, plasmid without LcrV, 5: L. plantarum, expressing LcrV, 6: L. plantarum expressing LcrV+lipid anchor (lipidation signal) OspA, 7: L. plantarum, expression LcrV+lipid anchor (lipidation signal) OspA with optimized codon usage of anchor (lipidation signal) sequence for Lactobacillus, 8: L. plantarum, expressing LcrV+modified lipid anchor (lipidation signal) (c17D) OspA with optimized codon usage of anchor (lipidation signal) sequence. LcrV (37 Kd) was detected using mAb 19.3.

FIG. 3 shows the percentage of mice with anti-OspA antibodies as a function of time following immunization.

FIG. 4 shows a characterization of the IgG subclass induced by oral immunization with L. plantarum expressing OspA (LpA) in comparison with the control (Lp) by ELISA. The figure indicates anti-OspA IgG isotype at termination.

FIG. 5 shows OspA-ELISA OD values from sera from mice immunized via oral administration of vaccine, two days before challenge with B. burgdorferi infected field ticks (day 66, see FIG. 7). The oral vaccines included Lactobacillus bacteria expressing OspA having a wild type leader (LpA) or a leader having the Cys-17 mutant (LpAc17d).

FIG. 6 shows protection by OspA vaccines of the invention (LpA, LpAα, LpA_{c17d}) against challenge by B. burgdorferi. FIG. 6A shows dark field microscopy (DFM) of cultures from heart and bladder. FIG. 6B shows PCR analysis of OspA and OspC in heart and bladder tissues. FIG. 6C shows PCR analysis of OspA and OspC in heart and bladder cultures. See Example V for a further discussion of this experiment.

LpA, vaccine antigen, Lactobacillus plantarum expressing OspA with wild type leader sequence; LpAα, Lactobacillus plantarum expressing OspA_{LFA}-, with wild type leader sequence; and LpAc17d, Lactobacillus plantarum expressing OspA with mutated leader sequence.

FIG. 7 shows an immunization protocol.

FIG. 8 shows LcrV-ELISA OD values from sera from mice immunized via oral administration of vaccine, three days before challenge (day 70). The oral vaccines included Lactobacillus bacteria without the expression vector (Lac-Lp), used as a negative control, a Lactobacillus bacteria expressing LcrV without the leader sequence (Lac-LcrV) and a Lactobacillus bacteria expressing LcrV having a wild type leader (Lac-LipLcrV) or a leader having the Cys-17 mutant (Lac-LipC17DLcrV).

### DESCRIPTION OF THE INVENTION

The present invention relates, e.g., to a non-pathogenic bacterium, e.g. a Lactobacillus bacterium, which expresses a polypeptide of interest that is operably linked, at its N-terminus, to a lipoprotein signal sequence from a surface-localized protein of a Borrelia bacterium, such as the outer surface protein A (OspA). A lipoprotein signal sequence is sometimes referred to herein as a “leader” sequence, a “signal” sequence, a “leader,” or a “leader peptide.” The non-pathogenic bacterium can be part of an immunogenic composition, e.g. a live bacterial vaccine. In embodiments of the invention, the leader sequence is operably linked to a polypeptide from a pathogen of interest, such as a bacterium, virus, fungus or parasite. In one embodiment, the polypeptide is from Yersinia pestis (Y. pestis), the etiologic agent for plague.

Among the advantages of the immunogenic compositions (e.g. vaccines) and methods of the present invention are that they are inexpensive, suitable for rapid immunization of large numbers of recipients, and easy to administer in resource-poor settings. For example, oral or nasal administered vaccines can be administered by untrained personnel, who can rapidly immunize large numbers of people. Vaccines comprising bacteria of the invention can be lyophilized and thus readily stored, e.g. in resource-poor settings which lack adequate means for refrigeration. Live vaccines present advantages in that the antigen is expressed in the context of an innately immunogenic form. One advantage of oral or nasal immunization with a live bacterial vaccine is that it can induce mucosal immunity as well as a protective systemic immune response. Live vaccines replicate and persist in the host, restimulating the host immune system and obviating the need for multiple doses; this provides an advantage with regard to patient compliance. Furthermore, a live bacterial vaccine eliminates the need to purify the antigen, thereby reducing costs; and it can be designed to deliver multiple antigens, reducing the number of times an individual must be vaccinated.

One aspect of the invention is a non-pathogenic bacterium (e.g., Lactobacillus),

which expresses a (one or more) recombinant polypeptide comprising a lipoprotein signal sequence from a surface-localized protein of Borrelia burgdorferi, or an active variant of the signal sequence, operably linked to a (one or more) heterologous polypeptide of interest,

and/or

which comprises an expressible polynucleotide encoding a recombinant polypeptide, wherein the polynucleotide encodes a lipoprotein signal sequence from a surface-localized protein of Borrelia burgdorferi, or an active variant thereof, which is operably linked to a heterologous polypeptide of interest and, optionally, wherein the polynucleotide encoding the signal sequence is modified so that the codon usage is optimized for expression in the non-pathogenic bacterium. Generally, the lipoprotein signal sequence is located at the N-terminal end of the recombinant polypeptide. A bacterium of the invention can be a bacterium that has been transformed (preferably stably transformed) with such an expressible polynucleotide, or progeny of such a transformed bacterium.

As used herein, the singular forms “a,” “an,” and “the” include plural referents unless the context clearly dictates otherwise. For example, “a” heterologous polypeptide of interest, as used above, means one or more heterologous polypeptides of interest, which can be the same or different.

Another aspect of the invention is an immunogenic composition comprising a bacterium of the invention. Preferably, the immunogenic composition is effective (e.g. is in a suitable form and amount) to elicit an immune (immunological) response in a host against the polypeptide. In one embodiment, the immunogenic composition is a vaccine that is effective to protect against infection by an organism (e.g. a pathogen) which bears the polypeptide of interest. The host is generally a vertebrate (e.g. pets, vermin, wildlife, livestock, or poultry). Preferably, the vertebrate is a mammal, such as a human. Other aspects of the invention include a pharmaceutical composition comprising a bacterium, immunogenic composition or vaccine of the invention and a pharmaceutically acceptable carrier; and a vaccine comprising a bacterium or immunogenic composition of the invention and, optionally, an adjuvant.

In one aspect of the invention, the bacterium is a gram positive bacterium, such as a lactic acid bacterium, particularly a strain of Lactococcus or Lactobacillus spp, such as Lactobacillus plantarum.

The leader (lipoprotein signal sequence) can be from any surface-localized protein of Borrelia burgdorferi, provided it contributes to the immunogenicity of the polypeptide to which it is operably linked. Without wishing to be bound by any particular mechanism, it is suggested that, at least in some embodiments of the invention, the leader is located at the N-terminus of the recombinant polypeptide; is processed (e.g., participates in lipidation and is cleaved from the linked heterologous polypeptide of interest); and the remaining lipidated polypeptide is localized at the surface of the bacterium. Therefore, in these embodiments, a polypeptide having an uncleaved N-terminal leader serves as an intermediate in the production of the processed, surface-localized polypeptide. A “surface-localized polypeptide,” as used herein, refers to a polypeptide that is in association with the surface of a bacterium, e.g. attached to or imbedded in a bacterial membrane, cell wall matrix or biofilm. The polypeptide may face either out from, or in toward, the bacterial cytoplasm. Without wishing to be bound by any particular mechanism, it is suggested that the leader may contribute to the immunogenicity of a heterologous polypeptide to which it is operably linked by allowing the heterologous polypeptide to become lipidated and/or to be surface-localized.

In one aspect of the invention, the leader sequence is a lipoprotein signal sequence from the OspA protein of Borrelia burgdorferi. This leader is sometimes referred to herein as “LipA” (Lipidation signal of ospA). In one embodiment, the OspA leader sequence comprises the amino acid sequence MKKYLLGIGLILALIAC (SEQ ID NO: 1) This polypeptide sequence can be encoded by any suitable nucleic acid, e.g. a nucleic acid having the sequence atg aaa aaa tat tta ttg gga ata ggt cta ata tta gcc tta ata gca tgt (SEQ ID NO:2). In another embodiment, the OspA leader sequence consists of the first 16 amino acids of SEQ ID NO:1. Such a leader, which lacks the C-terminal Cys residue of SEQ ID NO: 1, can lipidate a heterologous polypeptide to which it is operably linked at a Cys residue that is present at the N-terminus of the linked heterologous polypeptide.

In aspects of the invention, the polypeptide(s) of interest is from a bacterial, viral, fungal or parasitic pathogen. As used herein, the term a “polypeptide” from an organism includes a full-length protein or an immunogenic fragment of a full-length protein. In one embodiment of the invention, the polypeptide is from the bacterium, Yersinia pestis, e.g. is one or more of the polypeptides LcrV (e.g. serotype O:3), F1, Fla or YopD, or an immunogenic fragment thereof. In another embodiment, the polypeptide of interest is a B. Burgdorferi polypeptide other than OspA (e.g., OspB, OspC, etc).

A bacterium of the invention can be lyophilized, or in liquid form. Furthermore, the bacteria can be either live or dead, or combinations of live and dead bacteria can be used. For example, dead bacteria can be used for priming, followed by live bacteria for a boost, or vice-versa.

Another aspect of the invention is a method for eliciting an immune response to a polypeptide of interest in a vertebrate (e.g. a human), comprising administering to the vertebrate an effective amount of a bacterium, immunogenic composition or vaccine of the invention which expresses that polypeptide. An “effective amount,” as used herein, is an amount that is effective to achieve at least a measurable amount of a desired effect. For example, the amount may be effective to elicit an immune response, and/or it may be effective to elicit a protective response, against a pathogen bearing the polypeptide of interest. The bacterium, immunogenic composition or vaccine can be administered by a variety of routes, including orally or intranasally. In one embodiment, it is administered to a mucosal lining. It may be administered in a single dose or in two or more repeated doses. In embodiments of the invention, the bacterium, immunogenic composition or vaccine stimulates a systemic protective response, a mucosal protective response, or both.

Another aspect of the invention is a method for inhibiting the infectivity and/or pathogenicity of a pathogen in a subject, comprising administering to the subject an effective amount of a bacterium, composition or vaccine of the invention (e.g. wherein the heterologous polypeptide(s) is derived from the pathogen), under conditions in which the polypeptide is effective to elicit an immune response that inhibits the infectivity and/or pathogenicity of the pathogen. A heterologous polypeptide “derived” from a pathogen can take any of a variety of forms. For example, a full-length polypeptide can be cloned from the pathogen, or can be obtained from library of polypeptides that have been cloned from the pathogen. A polypeptide that has been “derived” from a pathogen can be a full-length polypeptide, or it can be an active fragment or variant of the full-length polypeptide. Examples of active fragments and variants of heterologous polypeptides are discussed elsewhere herein.

Another aspect of the invention is a method for delivering an immunogenic polypeptide to a subject in need thereof, comprising administering to the subject a bacterium, immunogenic composition or vaccine of the invention which expresses that polypeptide.

Another aspect of the invention is a method for enhancing an immune response in a host to a polypeptide of interest, comprising (a) generating an expressible polynucleotide which comprises a lipoprotein signal sequence from the OspA protein of Borrelia burgdorferi, or an active variant thereof, which is operably linked to the polypeptide of interest; and (b) introducing the expressible polynucleotide into a non-pathogenic bacterium (e.g. a Lactobacillus bacterium). When this bacterium is then introduced into a host, the immune reaction in the host is enhanced compared to the immune reaction elicited by the starting polypeptide (not operably linked to the signal sequence) by itself, and/or compared to the immune reaction elicited by the starting polypeptide (not operably linked to the signal sequence) when it is expressed in the non-pathogenic bacterium. The polynucleotide encoding the lipoprotein signal sequence may be modified so that its codon usage is optimized for expression in the non-pathogenic bacterium.

Another aspect of the invention is a method for making a non-pathogenic bacterium of the invention, comprising introducing into a suitable bacterium (e.g., a gram positive bacterium, such as a lactic acid bacterium, e.g. Lactobacillus) an expressible polynucleotide encoding a recombinant polypeptide which comprises a lipoprotein signal sequence from a surface-localized protein of Borrelia burgdorferi, or an active variant thereof, which is operably linked to the polypeptide(s) of interest. The polynucleotide encoding the signal sequence may be modified so that its codon usage is optimized for expression in the non-pathogenic organism. Preferably, the polynucleotide is introduced (e.g. transformed) stably.

Another aspect of the invention is a kit comprising a bacterium, immunogenic composition or vaccine of the invention. Optionally, the kit may comprise means for packaging the bacterium, immunogenic composition or vaccine and/or written instructions defining a vaccine regimen. The bacterium, immunogenic composition or vaccine may be in a lyophilized form or in liquid form. The kit may comprise a bacterium, immunogenic composition or vaccine of the invention and, optionally, an adjuvant.

In general, a bacterium of the invention comprises an expressible nucleic acid that encodes a recombinant polypeptide comprising a polypeptide of interest (e.g. a heterologous polypeptide) which is operably linked (generally at its N-terminus) to the N-terminal lipoprotein signal sequence of a surface-localized protein of Borrelia burgdorferi (B. burgdorferi, also sometimes referred to herein as Borrelia), such as the OspA protein. An “expressible nucleic acid,” as used herein, refers to a nucleic acid that can be expressed in a cell, e.g. transcribed and translated. For example, a nucleic acid comprising a coding sequence that is operably linked to an expression control sequence is an expressible nucleic acid. As used herein, the term “operably linked,” when referring to the linkage of a nucleic acid expression control sequence to a coding sequence, means that the expression control sequence is positioned in such a manner that it can regulate the expression of the coding sequence. The term operably linked, when referring to the linkage of a polypeptide leader (lipoprotein signal sequence) to a polypeptide of interest, means that the leader (signal sequence) is positioned in such a manner that it can enhance the immunogenicity of the linked polypeptide of interest.

The mechanism by which the leader (lipoprotein signal sequence) enhances the immunogenicity of the polypeptide of interest is not critical to the invention. In one embodiment of the invention, the leader (signal sequence) is fused to the polypeptide of interest to create a fusion polypeptide. The leader (signal sequence) is generally positioned at or near the N-terminus of the polypeptide of interest, but it may also be located at another position, provided it is able to enhance the immunogenicity of the polypeptide of interest.

A lipidation/processing reaction has been described for the intact OspA gene of B. burgdorferi. The primary translation product of the full-length B. burgdorferi OspA gene contains a hydrophobic N-terminal sequence, of 16 amino acids, which is a substrate for the attachment of a diacyl glyceryl to the sulflhydryl side chain of the adjacent cysteine (Cys) residue (at position 17). Following this attachment, cleavage by signal peptidase II and the attachment of a third fatty acid to the N-terminus occurs. The completed lipid moiety, a tripalmitoyl-S-glycerylcysteine modification, is termed Pam₃Cys (or is sometimes referred to herein as Pam(3)Cys or Pam3Cys). It has been suggested that the lipid modification allows membrane localization of proteins, with polypeptide portions exposed as immune targets. In addition to serving as targets for the immune response, Pam3Cys-modified proteins, such as OspA, have been reported to act as potent inflammatory stimulants though the toll-like 2 receptor mechanism (TLR2).

The present inventors show herein that by cloning an N-terminal signal peptidase II cleavage sequence (leader sequence) from the OspA protein of Borrelia burgdorferi adjacent to a polypeptide of interest, particularly a polypeptide that does not contain a classical leader (lipoprotein signal peptide) sequence, one can enhance the immunogenicity of the polypeptide of interest when it is expressed in, for example, a Lactobacillus bacterium. Without wishing to be bound by any particular mechanism, it is suggested that, at least in some embodiments of the invention, the enhancement of immunogenicity may come about because the cloned leader provides a “transgenic” site for lipid modification (Pam3Cys) of the polypeptide of interest, and this lipid modification is at least partially responsible for converting these polypeptides into more effective stimulants of the immune system. The leader sequence can thus serve as an adjuvant to enhance the immunogenicity of the polypeptide of interest.

The cloned leader sequence may be a wild type, 17 amino acid leader sequence from OspA, as represented by SEQ ID NO:1, which includes a Cys residue at position 17 that can become lipidated. This leader sequence can be cloned adjacent to (or near) a polypeptide of interest (e.g. a heterologous polypeptide) which lacks a classical signal peptide that is characteristic of a lipoprotein (sometimes referred to herein as a “lipoprotein signal sequence” or a “signal sequence”), or it can be substituted for such a classical signal peptide. In one embodiment of the invention, a polypeptide of interest which lacks a suitable Cys residue at its N-terminus is operably linked to an OspA leader that contains the C-terminal Cys residue at position 17 of SEQ ID NO: 1. In other embodiments of the invention, e.g. when the polypeptide of interest contains a suitable Cys at its N-terminus, it is not necessary to include the Cys at position 17 in the operably linked leader sequence. In these embodiments, an OspA leader containing the first 16 amino acids of SEQ ID NO: 1, but not the Cys residue at position 17, can be used.

The Borrelia burgdorferi genome contains over 150 other genes possessing the signal peptidase II cleavage sequence (leader sequence) required for a Pam3Cys modification to the amino terminus of the protein; the leaders of some of these genes can also be used in compositions and methods of the invention, provided the leader, when operably liked to a polypeptide of interest, is able to enhance the immunogenicity of the linked polypeptide.

In embodiments of the invention, a leader sequence may comprise, consist of, or consist essentially of the sequence represented by SEQ ID NO:1. “Consist essentially of,” as used herein with reference to amino acid sequences, refers to a sequence which is intermediate in length between the number of amino acid residues encompassed by the term “consisting of” and the longer length encompassed by the term “comprising.” Residues in addition to the residues encompassed by “consisting of” language do not affect the basic and novel characteristics (e.g., in the case of a leader of the invention, the ability to enhance the immunogenicity of a polypeptide to which it is fused) of the molecule encompassed by the “consisting of” language. For example, a suitable leader sequence can be truncated at the N-terminus of the 17 amino acid sequence of SEQ ID NO:1, e.g. by about 1-7 amino acids, leaving a leader of about 10-16 amino acids. In one embodiment, the leader sequence is truncated at the N-terminus by about 1-3 amino acids, leaving a leader of about 14, 15 or 16 amino acids. Alternatively, or in addition, a suitable leader sequence can comprise as many as about 15 additional amino acids (or more) that lie C-terminal to the Cys-17 residue of the OspA leader, or it can contain additional amino acids which do not correspond to the OspA sequence, provided that the amino acids added to the N-terminus of the processed polypeptide of interest do not interfere with the function of the polypeptide of interest. For example, one or two amino acids can be added to the C-terminal end. That is, the leader can consist of between about 10 and 36 amino acids, such as between about 14 and 29 amino acids. (All ranges provided herein include the end point values). Leaders that differ in size from the polypeptide represented by SEQ ID NO:1 can be active variants of that leader.

An “active variant” of a leader, as used herein, is a variant leader that retains the ability of the wild type leader to enhance the immunogenicity of the linked polypeptide. Suitable variants, in addition to the length variants discussed above, may comprise small deletions, insertions or substitutions compared to the wild type leader. In one embodiment, the variant contains one or more conservative amino acid substitutions. The variants may be naturally occurring (e.g., allelic variants or strain differences), or they may be introduced artificially, using conventional methods. A skilled worker can readily determine if a given leader, either wild type or variant, can enhance the immunogenicity of an operably linked polypeptide.

Guidance as to what sorts of variations can be tolerated in a leader can be obtained by analogy to disclosures in, e.g., the following references: Hayashi et al. (1990) J. Bioenerg. Biomembr. 22, 451-471; Loleit et al. (1994) Biol. Chem. Hoppe Seyler 375, 407-12; Offermanns et al. (1992) Biochem. J. 282, 551-7; Gonnet et al. (2004) Proteomics 4, 1597-613; Venema et al. (2003) J. Biol. Chem. 278, 14739-46; Reitermann et al. (1989) Biol. Chem. Hoppe Seyler 370, 343-52; or U.S. Pat. Nos. 6,143,872, 6,902,893, or 6,183,986.

For example, a consensus sequence has been identified at the C-terminal region of lipoprotein signal peptides at which the signal peptidase II cleavage takes place. The consensus sequence is sometimes represented as L-y-x-C, at positions −3 to +1 relative to the point of cleavage, wherein x and y are independent of each other, and each is a small amino acid, such as a small neutral amino acid (e.g., isoleucine, alanine, glycine, etc.). In order to maintain proper cleavage, these four amino acids would be expected to be relatively invariant, whereas changes might be better tolerated at other locations in the leader. Furthermore, sequences which have been identified as imparting immunogenic properties to small peptides which contain a Pam3Cys lipid modification can be used in conjunction with the present invention. See, e.g., Reitermann et al. (1989), supra. Alternatively, other amino acid sequences can be added or substituted for those in the leader, such as the 6 aminohexanoic acid described in Loleit et al. (1994), supra.

It is often desirable that a nucleic acid encoding a leader sequence be modified so that its codons are suitable for optimal expression in a bacterial host of interest. For example, ata codons are very rare in Lactobacilli. Thus, for optimal expression in Lactobacillus, one or more (e.g. all) of the ata codons of, e.g., the OspA leader from B. burgdorferi, can be changed, independently, to atc or att. Other variations in codon usage, for optimizing expression in Lactobacillus or any desired host, will be evident to the skilled worker.

As used herein, a “non-pathogenic” bacterium (e.g., non-invasive, commensal, or symbiotic) is one that does not cause a detrimental pathogenic effect to its host. Preferably, the bacterium is a naturally occurring, GRAS (“generally recognized as safe”), probiotic bacterium. However, in some embodiments, the bacterium may be an attenuated form of a pathogen.

In general, a bacterium used to generate an immunogenic composition of the invention can colonize the mucosal surface of an organ or tissue of interest. It is desirable (although not required) that a bacterial vaccine targeted against a particular pathogen can occupy the mucosal lining of an organ or tissue by which the pathogen is introduced into a subject. For example, in the case of an immunogenic bacterial composition targeted against an aerobically introduced pathogen, such as Yersinia pestis, it is desirable that the immunogenic bacterium can occupy the mucosal lining of a respiratory organ, such as the upper respiratory tract (e.g., oral cavity or larynx). However, bacteria that are introduced into other mucosa, e.g. intranasally, can produce protective mucosal immunity in the lung. In another embodiment, in which the pathogen is introduced into the subject via the digestive system, it is desirable that the immunogenic bacterium can occupy a mucosal lining of the gastrointestinal tract (e.g., the colon, cecum or upper intestine (jejunum, duodenum and ileum)). In another embodiment, in which the pathogen, such as an HIV virus, is introduced via the genitourinary tract system, it is desirable that the immunogenic bacterium can occupy a mucosal lining or the genitourinary tract (e.g., the cervix, vagina, penis, rectum or urinary tract). Many organisms that can colonize one type of mucosum can also colonize another type of mucosum (e.g., some bacteria which can colonize gastrointestinal mucosa are also able to colonize genitourinary mucosa, and vice-versa). Generally, if an immunogenic bacterium of the invention is targeted to a particular mucosal lining in the body, it is preferred that the immunogenic bacterium occupies that desired site and does not invade other areas of the body, such as the blood or heart. It is noted that immunization at any mucosal tissue site can elicit an immune response at all other mucosal sites. Thus, for example, immunization in the gut can elicit mucosal immunity in the upper airways and vice-versa.

Among the bacteria that can be used in the invention are bacteria which naturally inhabit the mucosum of interest, or which have been manipulated (e.g., adapted) so that they can colonize the mucosum. Exemplary methods for adapting such bacteria are discussed below. A bacterium that can “colonize” a mucosum is one that can compete with the preexisting microflora and take up residence in the mucosum. A “mucosal membrane” or a “mucosal surface” refers to a tissue layer found lining various tubular cavities of the body such as the oropharynx, lung, small intestine, large intestine, rectum, penis, vagina, mouth, uterus, etc. It is composed of a layer of epithelium containing numerous unicellular mucous glands and an underlying layer of areolar and lymphoid tissue, separated by a basement membrane. This membrane is typically colonized by a variety of bacteria even when the host is healthy.

Non-pathogenic bacteria of the invention may be any of a variety of types. Preferably they are strains which exhibit favorable growth and colonization properties, and which can be efficiently, and preferably stably, transformed with recombinant DNA constructs. By “favorable growth and colonization properties” is meant that the bacterium can efficiently colonize a mucosal lining and can continue to grow and/or remain attached to the mucosal lining to the extent necessary to generate an immune response against the polypeptide of interest. In some cases the bacterium is a genetically engineered version of a species that naturally inhabits the mucosum which is being colonized. In some cases it is a modified version of a strain that has been previously administered to humans as a probiotic and thereby known to be a good colonizer and non-pathogenic.

Humans are inhabited by over 1000 different species of bacteria which inhabit and/or can colonize normal healthy mucosa, and which can be used in methods of the invention (see, e.g., Guamer et al. (2003) Lancet 361, 512-9; Salminen et al. (1995) Chemotherapy 41 Suppl 1, 5-15; and Galask, R. P. (1988) Am J Obstet Gynecol 158, 993-5). Several specific examples of suitable bacteria are described below, but a skilled worker may recognize appropriate ways to modify any particular disclosure herein so as to be applicable to additional species, strains and isolates of bacteria. Bacteria can be used which naturally exhibit desired growth and colonization behavior. Alternatively, bacteria can be manipulated, using conventional procedures, to enhance their ability to colonize a mucosal surface. For example, a first method involves repetitively selecting for rapid colonizing bacteria on animal or human mucosal layers. For example, one applies a wild type bacterial strain to a mucosal surface and repetitively isolates and in vitro cultures bacteria, returning at each step to the mucosal surface. Ultimately, a bacterium with an enhanced colonizing ability is obtained. A second method involves expression of fusion proteins on the surface of recombinant bacteria. The fusion protein consists of a host-binding domain linked to a polypeptide of interest. The host-binding domain will allow the bacteria to bind to certain determinants (protein or carbohydrate) on a selected host mucosal surface with high affinity, thus conferring the bacteria a survival advantage over the resident microflora. In addition, one can use bacterial strains known to be non-pathogenic and efficient colonizers by virtue of their use as probiotics, which are live bacteria which when administered in adequate amounts confer health benefits on the host. Typically, probiotic bacteria have demonstrated safety in human use, survival in the intestine, adhesion to mucosa, and at least temporary colonization of the gut.

Suitable bacteria include non-pathogenic and attenuated mucosal bacteria. In one embodiment, the non-pathogenic bacterium is a gram negative bacterium, such as a strain of E. coli. Suitable E. coli strains include, for example, the Nissle 1917 strain, which is widely used as a probiotic in Europe. Other suitable gram negative bacteria include attenuated forms of Enterobacteria (e.g. Salmonella, Shigella or Yersinia), Vibrionaceae, Francisellaceas, Legionallales, Psuedomonadaceae or Pasteuralaceae. In another embodiment, the bacterium is a gram positive bacterium, e.g. an attenuated form of Listeria, Staphylococcus or Streptococcus. Among the types of bacteria which can be used are non-pathogenic forms of Bacillus (which has been employed as a probiotic (Hoa et al. (2000) Appl Environ Microbiol 66, 5241-7)); Staphylococcus sps, S. epidermidis, S. aureus, and Neisseria sps, all of which naturally inhabit at least the nasal/oral pharynx of healthy individuals; Corynebacterium sps, which naturally inhabits vaginal mucosa; and mycobacteria. Furthermore, vectors are also available for Bifodobacteria, which are among the most common bacteria in the human intestine (van der Werf et al. (2001) J Agric Food Chem 49, 378-83).

In a preferred embodiment of the invention, the bacterium is a lactic acid bacterium, such as a Lactococcus or Lactobacillus bacterium. Suitable Lactococcus bacteria include, e.g., Lactococcus cremoris and Lactococcus lactis, which is a non-pathogenic gram positive bacterium frequently used to produce fermented foods, and which has been engineered to secrete interleukin-10 as a treatment for murine colitis (Steidler et al. (2000) Science 289, 1352-5); Lactococci generally do not colonize the mucosum of a host, and thus must be administered to a host more than once, preferably continuously, until a desired immunogenic effect is achieved. Other suitable lactic acid bacteria include, e.g., Pedicoccus, Enterococcus, Propionibacterium. Streptococcus thermophilus, or Staphylococcus carnosus.

In a most preferred embodiment of the invention, the bacterium is a Lactobacillus bacterium. The “generally recognized as safe” status of dietary lactic acid bacteria such as Lactobacillus spp. render them particularly attractive as mucosal vaccine carriers. Some Lactobacillus strains are also reputed to exert beneficial health properties and have been intensively studied for probiotic applications (see, e.g., Reveneau et al. (2002) Vaccine 20, 1769-77). Lactobacilli bacteria maintain a sophisticated, non-invasive ecology within the host and the capacity of some strains of Lactobacilli to enhance immune responses against particular antigens or natural immuno-adjuvanticity has been demonstrated (Gerritse et al. (1990) Res. Microbiol. 141, 955-62; Perdigon et al. (1991) J. Dairy Res. 58, 485-96; Link-Amster et al. (1994) FEMS Immunol Med Microbiol 10, 55-63; Pouwels et al. (1996) J. Biotechnol. 44, 183-92).

Lactobacillus isolates with favorable growth and colonization properties, which can be transformed efficiently with heterologous DNA and which express a heterologous polypeptide of interest in a suitable form and amount to elicit a desired immunogenicity, are suitable for use in the present invention. Suitable strains can be selected from, e.g., Lactobacillus sps, L. plantarum, L. reuteri, L. johnsonii, L. casei (e.g., L. casei 393, L. casei ss rhamnosus, L. casei ss alactosus), L. crispatus, L. fermentum, L. salivarius, L. catenaforme, L. minutus, L. gasseri, L. acidophilus, L. jensenii, L. helveticus, L. amylovorus, L. gallinarum, L. murinus, L. animalis, L. zeae, L. buchneri, and L. brevis. L. plantarum is particularly well-suited for oral administration or bacterial vaccines. In particular, L. plantarum strain 256 has been reported by one of the present inventors to be especially well-suited. See, e.g., J. F. Seegers (2002) Trends in Biotechnol. 20, 508-15. A variety of other suitable L. plantarum will be evident to the skilled worker; these include, e.g., L. plantarum 80, and L. plantarum strains LMG 9211, LMG 1284, LMG 6907, LMG 8155, LMG 9205, LMG 9206, LMG 9208, LMG 9209, LMG 9210. LMG 9212, LMG 11405, LMG 11460, LMG 8095, LMG 8027, LMG 12167, LMG 13556, LMG 17552, LMG 18021, LMG 18023, LMG 18024, LMG 18027, LMG 18095; 386, 299, 105 or 275 (see Molin et al. (1993) J. Appl. Bacteriol. 74, 314), 299v (see WO 96/29083); So5, 36.sup.E, 95, 120 or 44 (see Johannson et al (1995) Int. J. Syst. Bacteriol. 45, 670-675), 79, 107, 98, 53, 97, 101 or 125 (see Johansson et al (1995) Int. J. Food. Micro. 25, 159), CH, ATCC 8041, ATCC 10012, ATCC 10776, WCFS, DF66 IIIa, DF66spez.-IVa, and/or the L. plantarum strains available from the Japanese Collection of Micro-organisms under the accession numbers: 8341, S342, 8343, S344, 8345, 8346, 8347 and/or 8348. For the delivery of bacterial vaccines to the vagina, e.g. for an AIDS vaccine, a natural vaginal isolate of Lactobacillus jensenii—Lactobacillus jensenii strain 1153—that exhibits favorable growth and colonization properties, can be used. See, e.g., Chang et al. (2003) Proc Natl Acad Sci USA 100, 11672-7 and US Pat. Pub. 20030228297.

As noted above, a bacterium of the invention may comprise a recombinant polynucleotide which encodes a polypeptide of interest operably linked to a leader sequence of the invention, wherein the coding sequences of the recombinant polynucleotide are operably linked to an expression control sequence. The polynucleotide can have been introduced into the bacterium (or an ancestor thereof) by transfection, transformation, or the like.

As used herein, the term “expression control sequence” refers to a polynucleotide sequence that regulates expression of a polypeptide coded for by a polynucleotide to which it is functionally (“operably”) linked. Expression can be regulated at the level of the mRNA (transcriptionally or post-transcriptionally) or polypeptide (translationally or post-translationally). Thus, the term expression control sequence includes mRNA-related elements and protein-related elements. Such elements include promoters, domains within promoters, upstream elements, enhancers, ribosome binding sequences, transcriptional terminators, etc. An expression control sequence is operably linked to a nucleotide sequence (e.g., a coding sequence) when the expression control sequence is positioned in such a manner to effect or achieve expression of the coding sequence. For example, when a promoter is operably linked 5′ to a coding sequence, expression of the coding sequence is driven by the promoter. Examples of promoters that can be used to drive expression in host bacteria include promoters of endogenous protein genes or genes of phage which can infect the bacteria. In the case of translational signals, such as a ribosome binding sites, the control element is typically inserted in between the promoter and the start point of translation. Many potent translational control sequences are available from highly expressed chromosomal loci or from suitable bacteriophage.

In one embodiment of the invention, a sequence encoding a fusion polypeptide (a polypeptide of interest that is operably linked to a suitable leader) is expressed under the control of a constitutive promoter. When such elements are present, they lead to constitutive high level expression of the antigenic polypeptide of interest. Suitable constitutive promoters for use in Lactobacillus include, e.g., the promoter of the lactate dehydrogenase (LDH) gene of L. plantarum or L. casei 393, the promoter of the S-layer protein (SlpA) of L. acidophilus, or the P₅₉ (van der Vossen et al. (1992) Appl. Environ. Microbiol. 171, 3656-66) or P₂₃ (Elliot et al. (1984) Cell 36, 211-219) promoters. Alternatively, promoters that are induced in the conditions in which the host bacteria colonizes the mucosa can be employed. Suitable inducible promoters include, e.g., L. amylovorus amylase promoter (amyA) or the L. pentosus xylose promoter (xylA, Lokman et al. (1994) Mol. Gen. Genet. 245, 117-5), the Bacillus amylase (Weickert et al. (1989) J. Bacteriol. 171, 3656-66) or xylose (Kim et al. (1996) Gene 181, 71-76) promoters as well as the Lactococcus nisin promoter (Eichenbaum et al. (1998) Appl. Environ. Microbiol. 64, 2763-2769). In addition, acid or alkaline-induced promoters can be used. For example, promoters that are active under the relatively acidic conditions of the vagina (e.g., those described in U.S. Pat. No. 6,242,194) can be used. Alternatively, promoters can be used that are induced upon changes in the vagina in response to semen. For example, alkaline-induced promoters can be used to induce expression in response to the increased alkaline conditions of the vagina resulting from the introduction of semen. Promoters regulated by iron are activated in the gastrointestinal tract.

Methods of making recombinant constructs, e.g. in which a sequence encoding a polypeptide of interest is operably linked to an expression control sequence, as well as other molecular biology methods used in conjunction with the present invention, are conventional. See, e.g., Sambrook, et al. (1989), Molecular Cloning, a Laboratory Manual, Cold Harbor Laboratory Press, Cold Spring Harbor, N.Y.; Ausubel et al. (1995). Current Protocols in Molecular Biology, N.Y., John Wiley & Sons; Davis et al. (1986), Basic Methods in Molecular Biology, Elseveir Sciences Publishing, Inc., New York; Hames et al. (1985), Nucleic Acid Hybridization, IL Press; Dracopoli et al. (current edition) Current Protocols in Human Genetics, John Wiley & Sons, Inc.; and Coligan et al. (current edition) Current Protocols in Protein Science, John Wiley & Sons, Inc.

Expression vectors have been designed for Lactobacillus spp. for targeted delivery of antigens. See, e.g., Pouwels et al. (2001) Methods Enzymol 336, 369-89; J. F. Seegers (2002), supra; or Mercenier et al. (2000) Curr Issues Mol Biol 2, 17-25. These expression systems are based on plasmids that have a cassette like structure that allows easy exchange of functions such as promoter sequence, secretion signal and selection marker. See also Shaw et al. ((2000), Immunology 100, 510-8; Reveneau et al. (2002), supra; Gerritse et al. (1990), supra; Pouwels et al. (1996), supra; Maassen et al. (1999) Vaccine 17, 2117-28; Pavlova et al. (2002) Plasmid 46, 182-92; Schepplet et al. (2002) Vaccine 20, 2913-20; Grangette et al. (2002) Vaccine 20, 3304-9; Rush et al. (1997) Appl Microbiol Biotechnol 47, 537-542; US patent application serial number 2005/0176788.

The expressible nucleic acids of the invention can be introduced into a bacterial cell by any of a variety of conventional methods, e.g. on a high copy plasmid; in a phage vector; or on a low copy number plasmid or stably integrated into a chromosomal integration site in conjunction with a strong promoter. Methods to introduce the expressible nucleic acids of the invention into bacterial cells will be evident to the skilled worker. The most common are chemical transformation, electroporation, and infection or transduction with a phage vector.

In general, a plasmid contains a selectable marker, such as resistance to an antibiotic, which is used to select for the plasmid and to maintain it in the cell. A large number of suitable selectable markers are known in the art, as are methods employing them. If desired, a cell comprising a plasmid which bears an antibiotic resistance gene can be introduced into a subject along with the antibiotic, in order to facilitate the establishment of the bacterium in the mucosum.

However, because antibiotic resistance markers might be transferred to opportunistic human pathogens such as Staphylococci and Enterococci, non-pathogenic bacteria of the invention may be modified to lack such antibiotic resistance markers when designated for use in the clinic. In this case, constructs of the invention can be stably integrated into the chromosome of a bacterium, so that a resistance marker is not required. A resistance marker that is used to select a stable transformant can be removed after the stable transformant is obtained by a variety of conventional genetic methods. Alternatively, a construct lacking a resistance marker can be introduced into the chromosome by homologous recombination.

Methods for inserting a sequence of interest into a bacterial genome in a stable fashion are conventional. For example, a number of bacteriophage vectors have been developed for use in different bacteria. A bacteriophage vector based on the temperate bacteriophage phi adh can be used (see, e.g., Raya et al. (1992) J. Bacteriol. 174, 5584-5592 and Fremaux et al. (1993) Gene 125, 61-66). This vector undergoes site-specific integration into the host chromosome at defined phage (attP) and bacterial (attB) attachment sites. Similarly, Lactobacillus-specific bacteriophage can be used to transduce vectors or other polynucleotides into the Lactobacillus chromosome. Lactobacillus-specific phage include mv4 (Auvray et al. (1997) J. Bacteriol., 179, 1837-1845), phi adh (Fremaux et al. (1993) Gene 126, 61-66), phi gle (Kakikawa et al. (1996) Gene 175, 157-165, and those belonging to Bradley's groups A or B in vaginal lactobacillus isolates (Kilic et al. (2001) Clin. Diagn. Lab. Immunol. 8, 31-39).

A variety of potentially antigenic (heterologous) polypeptides are encompassed by the invention. As used herein, the terms “polypeptide,” “peptide,” and “protein” are used interchangeably to refer to a polymer of amino acid residues. These terms as used herein encompass amino acid chains of any length wherein the amino acid residues are linked by covalent bonds. A full-length or nearly full-length polypeptide may be used in compositions and methods of the invention; or the polypeptide may be an immunogenic peptide fragment of a full-length protein. An “immunogenic peptide or fragment,” as used herein, refers to a peptide or fragment that retains one or more epitopes of the full-length molecule (and therefore the ability to elicit an immune response to the peptide or fragment). A skilled worker will recognize how to generate suitable immunogenic fragments e.g. based on known properties of the polypeptides. “Antigenic polypeptides or peptides,” as used herein, includes polypeptides or peptides which have been modified post-translationally, e.g. glycopeptides.

The terms “nucleic acid” and “polynucleotide” are also used interchangeably herein.

An immunogenic polypeptide of the invention can be used to inhibit or prevent any step in pathogenesis, including, e.g., initial infection of a naive host by the pathogen; continuing reinfection of a chronically infected host by the pathogen; detrimental biochemical, physiological, and immunological effects caused by infection with the pathogen; and spread of the pathogen to other hosts.

The pathogens which can be inhibited by methods of the invention include, e.g., bacteria, viruses, fungi and parasites.

In one embodiment of the invention, the pathogen which is inhibited is a bacterium. Among the bacteria that can be inhibited by methods of the invention are Yersinia pestis; bacteria which cause sexually transmitted diseases, including, e.g., Neisseria gonorrhoeae (gonorrhea), Treponema palladium (syphilis), and Chlamydia trachomatis (chlamydia); and a variety of other pathogenic bacteria, including Clostridiun spp, Corynebacterium spp, Bacillus anthracis, Streptococcus pneumoniae, Streptococcus mutans, Streptococcus sobrinus, Streptococcus equi, Streptococcus pyogenes, Erysipelothrix rhusiopathiae, Mycobacterium tuberculosis and Borrelia burgdorferi. The skilled worker will recognize a wide variety of suitable polypeptides, or active fragments or variants thereof, from any pathogen of interest, which can be used to generate an immunogenic bacterial composition that can elicit an immune response against the pathogen.

In another embodiment, the pathogen which is inhibited is a virus. Among the many viruses which can be inhibited by the methods of the invention are rotavirus, Norwalk agent, papillomavirus, adenovirus, respiratory syncytia virus, corona virus, cytomegalovirus, coxsackievirus, echovirus, hepatitis A virus, hepatitis B virus, hepatitis C virus, rhinovirus, human immunodeficiency virus, poliovirus and other picornaviruses, smallpox virus, Epstein-Barr virus, influenza virus, parainfluenza virus, and herpes simplex virus.

In another embodiment of the invention, the pathogen which is inhibited is a fungus, such as Candida or Aspergillus spp.

In another embodiment of the invention, the pathogen which is inhibited is a parasite, such as a worm or other helminth, or a protozoa.

Any antigen can be used to which one wishes to elicit an immune response in a host. Among the antigens which can be used are: Mycobacterium leprae antigens; Rickettsia antigens; Coxiella antigens; malaria sporozoite and merozoite proteins, such as the circumsporozoite protein from Plasmodium berghei sporozoites; diphtheria toxoids; tetanus toxoids; Leishmania antigens; Salmonella antigens; E. coli antigens; Listeria antigens; Borrelia antigens, including the OspA, OspB, OspC, dbpA, dbpB, Fla, VlsE and BBK32 antigens of Borrelia burgdorferi; Franciscella antigens; Mycobacterium africanum antigens; Mycobacterium intracellulare antigens; Mycobacterium avium antigens; Treponema antigens; Schistosome antigens; Filaria antigens; Pertussis antigens; Staphylococcus antigens; and parainfluenza virus antigens; measles virus antigens; Bordatella antigens; Hemophilus antigens; Streptococcus antigens, including the M protein of S. pyogenes and pneumococcus antigens such as Streptococcus pneumoniae antigens; mumps virus antigens; hepatitis virus antigens; Shigella antigens; Neisseria antigens; rabies antigens; polio virus antigens; Rift Valley Fever virus antigens; dengue virus antigens; measles virus antigens; rotavirus antigens; Human Immunodeficiency Virus (HIV) antigens, including the gag, pol, and env proteins; respiratory syncytial virus (RSV) antigens; snake venom antigens; human tumor antigens; and Vibrio cholera antigens. Other suitable antigens include a Borrelia antigen, e.g., OspA, OspC, OspB, OspD; a pneumococcal antigen, e.g., PspA; an influenza (Flu) antigen such as HA; a pertussis or whooping cough antigen such as the pertussis 69 KD polypeptide; a hepatitis antigen, e.g., hepatitis B antigen such as hepatitis B surface antigen; a Helicobacter pylori antigen such as urease; a rabies virus antigen, e.g., rabies G antigen; a flavivirus antigen, e.g., a Japanese encephalitis virus, Dengue virus or yellow fever virus antigen; a chicken pox virus antigen; a diphtheria antigen; a C. tetani antigen, e.g., tetanus toxoid; a mumps virus antigen; a measles virus antigen; a malaria antigen; a herpes virus antigen, such as an alphaherpesvirus, betaherpesvirus or gammaherpesvirus antigen, e.g., a herpes virus glycoprotein, for instance an equine herpesvirus antigen, e.g., gp13, gp14, gD, gp63, or gE, a pseudorabies virus antigen, e.g., gp50, gpII, gpIII, gpI, a herpes simplex virus antigen, e.g., gC, gD, a bovine herpes virus antigen, e.g., gI, a feline herpes virus antigen, e.g., gB, an Epstein-Barr virus antigen, e.g., gp220, gp340, or gH, or a human cytomegalovirus antigen, e.g., gB; a human immunodeficiency virus antigen, e.g., gp160 or gp120; a simian immunodeficiency virus antigen; a bovine viral diarrhea virus antigen; an equine influenza virus antigen; a feline leukemia virus antigen; a canine distemper virus antigen, e.g., HA or F glycoproteins; a canine adenovirus antigen, e.g., canine adenovirus type 2 antigen; a canine coronavirus antigen; a canine parainfluenza antigen; a canine parvovirus antigen; a Hantaan virus antigen; an avian influenza virus antigen e.g., a nucleoprotein antigen; a Newcastle Disease virus antigen, e.g., F, HN; an antigen of rous associated virus, e.g., an RAV-1 envelope antigen; an infectious bronchitis virus antigen, e.g., a matrix antigen or a preplomer antigen; an infectious bursal disease virus antigen; a cholera antigen; a tumor associated antigen; a feline immunodeficiency virus antigen; a foot-and-mouth disease virus antigen; a Marek's Disease Virus antigen; a Staphylococci antigen; a Streptococci antigen; a Haemophilus influenza antigen, e.g., group b polysaccharide-protein conjugates; a papilloma virus; a poliovirus antigen; a rubella virus antigen; a poxvirus, such as smallpox antigen, e.g., vaccinia; a typhus virus antigen; a typhoid virus antigen; a tuberculosis virus antigen; an HTLV antigen; or, other bacteria, virus or pathogen antigen, such as a bacterial or viral surface antigen or coat protein. In one embodiment, the antigen is at least one polypeptide which includes an epitope that is recognized by cytotoxic T lymphocytes induced by an HIV polypeptide or derivative thereof. The polypeptide may be an HIV polypeptide or derivative thereof. HIV polypeptides may include HIV-1-gp120; HIV-1-gp 41; HIV-1-gp 160; HIV-1-pol; HIV-1-nef; HIV-1-tat; HIV-1-rev; HIV-1-vif; HIV-1-vpr; HIV-1-vpu; HIV-1-gag; HIV-2gp120; HIV-2-gp 160; HIV-2-gp 41; HIV-2-gag; HIV-2-pol; HIV-2-nef; HIV-2-tat; HIV-2-rev; HIV-2-vif; HIV-2-vpr; HIV-2-vpu; and HIV-2-vpx.

Many of the antigens discussed herein are “heterologous” to the leader to which they are operably linked, and much of the discussion refers to such heterologous polypeptides of interest. A “heterologous” polypeptide, as used herein, refers to a polypeptide that is not normally operably linked to the leader in question. The heterologous polypeptide may be from the same or a different species as the leader. For example, a leader from the OspA gene of Borrelia burgdorferi that is operably linked to an antigen from Borrelia burgdorferi other than the OspA polypeptide is considered to be operably linked to a “heterologous” polypeptide.

A preferred target for a vaccine of the invention is Y. pestis. The Y. pestis genome has been completely sequenced along with its three virulence plasmids (Parkhill et al. (2001) Nature 413, 523-7). The alleles found in Y. pestis are isosequential to alleles found in Y. pseudotuberculosis. The genome is 4.63 megabases in size, with the three plasmids of 9.6 kb, 70.3 kb, and 96.2 kb in size. The three plasmids code for important virulence factors (Parkhill et al. (2001), supra; Cornelis et al. (2002) J Cell Biol 158, 401-8). All three pathogenic Yersinia—Y. pestis, Y. pseudotuberculosis and Y. enterocolitica—share the 70 kb plasmid which encodes the genes for the type III secretion system; LcrV; and a series of Yersinia outer surface proteins (Yops) required for pathogenicity (Perry et al. (1986) Infect. Immun. 54, 428-34; Nilles et al. (1997) J. Bacteriol. 179, 1307-16; Pettersson et al. (1999) Mol. Microbiol. 32, 961-76; Fields et al. (1999) Infect. Immun. 67, 4801-13). Both the 10 kb plasmid encoding plasminogen activator protease (Pla) and the 100 kb plasmid encoding F1 capsular antigen are unique to Y. pestis. LcrV was one of the first Y. pestis virulence antigens identified (Burrows et al. (1956) Nature 177, 426-427; Burrows et al. (1958) Br. J. Exp. Pathol. 39, 278-91). Along with at least fourteen different Yops (Cornelis et al. (1998) Microbiol. Mol. Biol. Rev. 63, 1315-52), they play a key role in the pathogenesis of Yersinia infection (Nilles et al. (1997), supra; Perry et al. (1997) Clin. Microbiol. Rev. 10, 35-66; Pettersson et al. (1999), supra; Fields et al. (1999), supra).

For vaccines directed against pathogenic Y. pestis, a variety of virulence factors expressed by the pathogen can be used as immunogenic polypeptides, either individually or in various combinations. Among the most useful polypeptides that can be used to generate antigenic bacteria of the invention are the low calcium response V protein (LcrV), the F1 capsular antigen (F1) and the Yersinia outer surface protein D (YopD). One embodiment of the invention is a multivalent vaccine containing, e.g., F1, two or three types of LcrV and YopD. Each of the polypeptides in a multivalent vaccine may be fused, independently, to a suitable leader. Alternatively, or in addition, the polypeptides may be cloned in tandem (in frame) and preceded by a suitable leader sequence.

### LcrV:

There are two well recognized evolutionary distinct types of the LcrV in Yersinia spp. One is expressed by Y. pseudotuberculosis serotype O:8 and the other is expressed by Y. pestis serotype O:3, Y. pseudotuberculosis serotype O:9 and Y. enterocolitica serotype O:5,27. Antibodies to LcrV are protective against Y. pestis expressing the same V antigen used to induce those antibodies but not against Y. pestis expressing the other V antigen serotype. An additional V antigen (V-Yp) expressed by some strains of Y. pestis has been more recently described, but details about this third LcrV are not well-understood. Thus, there are three, potentially interchangeable by genetic engineering, LcrV types, two of which are not cross protective.

Lawton et al. demonstrated that anti-LcrV antibodies provided protection against plague in mice (Lawton et al. (1963) J. Immunol. 91, 179-84). Other studies have verified that polyclonal antibodies raised against highly purified LcrV as well as a monoclonal antibody that recognizes the central domain of LcrV conferred passive protection against experimental infections with Y. pestis and Y. pseudotuberculosis. Reports suggest that active immunization with recombinant LcrV is protective in both experimental subcutaneous and aerosol challenge, and that immunization with LcrV protects mice against pneumonic and bubonic plague caused by FI+ and F1− strains of Y. pestis. Protective anti-LcrV antibodies neutralize Y. pestis in multiple ways, only one of which involves blocking Yop translocation.

The three dimensional structure of LcrV is known (see the PDB database). Monoclonal antibodies against F1 and LcrV have been reported to show synergy when administered prophylactically and as a therapy 48 hours after infection (Hill et al. (2003) Infection and Immunity 71, 2234-2238). The amino acids that determine Toll-like receptor 2 dependent IL-10 induction and virulence have been identified by Sing et al. (205) Proc Natl Acad Sci USA 102, 16049-54 and Overheim et al. (2005) Infection and Immunity 73, 5152-5159. LcrV epitopes recognized by CD4-T-cells are known (Parent et al. (2005) Infection and Immunity 73, 2197-2204.

### F1:

F1 antigen is unique to Y. pestis. This antigen is encoded on the 100 kb plasmid and like other bacterial capsular antigens has anti-phagocytic activity. It has a molecular weight of 15.5 kDa and aggregates to form a large gel-like capsule. Four genes, the structural gene for F1, cafl, and three associated genes, cafM, cafA, and cafR, enable the formation of this gel like capsule. F1 induces a protective immune response when injected into mice. Polymeric recombinant F1 and monomeric recombinant F1 are equally immunogenic but the polymeric F1 induces a significantly better protective response.

### YopD:

Together with the Ysc secretion machinery, the Yops form an integrated system that allows extracellular Yersinia to neutralize anti-bacterial functions in leukocytes. Yops are delivered when extracellular Yersiniae attach to the surface of eukaryotic cells. YopB, YopD and LcrV are required for translocation of effector Yops across the eukaryotic cell membrane. In addition, Yersinia has a contact-dependent pore forming activity that requires LcrV, YopD and YopB. YopB and YopD contain hydrophobic domains indicative of transmembrane proteins and both proteins can bind to LcrV. These observations have led to the suggestion that LcrV, YopB and YopD assemble on the tip of the secretion machinery and form a translocation pore that inserts in the host membrane. YopD is secreted prior to immune cell contact into the extracellular milieu after being induced by the presence of glutamate or albumin. Mice vaccinated with YopD and challenged with a virulent nonencapsulated isogenic Y. pestis strain were protected (Andrews et al. (1999) Infect. Immun. 67, 1533-7). This indicates that in the absence of a capsule that YopD is available to neutralizing antibodies. Without wishing to be bound by any particular mechanism, it is suggested that, given the accessory function of YopD in facilitating delivery of other Yops into their eukaryotic targets, antibody to YopD may interfere with this function.

In another embodiment, the target of a vaccine is not a pathogen, but is a cancer cell. Vaccines of the invention can be used to inhibit or treat tumor cells, and/or to control the growth, rate of invasion or survival of tumor cells. Suitable antigens for this use will be evident to the skilled worker. In another embodiment, the target is a gamete, and the antigenic bacterium may thus be used as a birth control method or anti-fertility treatment. Again, suitable antigens for this use will be evident to the skilled worker.

Active variants of any of the polypeptides of interest (e.g., heterologous polypeptides) discussed herein (including peptide fragments of larger proteins) are included in the invention, provided that the altered polypeptide retains at least one epitope of the unaltered (e.g., wild type) polypeptide, e.g., provided that it can elicit an immune response in a host. It is an accepted practice in the field of immunology to use fragments and variants of protein immunogens as vaccines and immunogens, as all that is required to induce an immune response to a protein may be a small (e.g., about 8 to 10 amino acid) region of the protein. The variant can have the sequence of a naturally occurring polypeptide, or it can have a variant of the sequence of a naturally occurring polypeptide. Suitable variants may comprise small deletions, insertions or substitutions compared to the wild type protein; the variant may contain one or more conservative amino acid substitutions. The variants may be naturally occurring (e.g., allelic variants or strain differences), or they may be introduced artificially, using conventional methods. A skilled worker can readily determine if a given polypeptide, either wild type or variant, contains a desired epitope.

Under suitable conditions, the administration to a subject of a bacterium of the invention elicits an immunogenic response against a pathogen of interest. “Suitable conditions,” as used herein, include the presence of regulatory elements that allow effective amounts of the polypeptide to be produced and to elicit an immune response. “Suitable conditions” also include a physiological environment which is conducive to the expression of the polypeptide. Such conditions are found, e.g., in the various mucosa to which the bacteria are administered. For example, suitable conditions for expressing an inducible promoter include a physiological environment in which an agent is present that induces the promoter. Suitable conditions may also include the presence of an amount of the bacterium that is sufficient to compete effectively with resident bacteria and to colonize the mucosal surfaces of an infected individual, thereby allowing the production of an immune response.

One aspect of the invention is a method to elicit an immune response to a polypeptide of interest in a vertebrate, comprising administering to the vertebrate an effective amount of a bacterium, immunogenic composition or vaccine of the invention which expresses that polypeptide. An “immune response,” as used herein, includes the following types of response, among others: circulating antibodies (primarily IgG) to neutralize toxins, promote complement-mediated cytolysis, opsonize extracellular bacteria for phagocytosis (with or without complement), or mediate antibody dependent cellular cytotoxicity towards pathogens or infected cells; cell-mediated immunity (CMI) including cytotoxic lymphocytes (CTL); and mucosal antibodies, primarily secretory Immunoglobulin A (SIgA)] to neutralize toxins and prevent pathogen colonization or penetration of mucosal surfaces. In one embodiment, to protect against a pathogen such as an aerosolized Y. pestis, a vaccine induces both protective systemic IgG and mucosal IgA antibodies.

An antibody “specific for” a polypeptide includes an antibody that preferentially recognizes a defined sequence of amino acids, or epitope, either present in the full length polypeptide or in a peptide fragment thereof. In one embodiment, the antibody is a neutralizing antibody. By “neutralizing” is meant herein that binding of an antibody to a pathogen or its receptor inhibits or prevents infection of the host by the pathogen.

Another aspect of the invention is a method for inhibiting infection by, or a pathogenic activity of, a pathogen (e.g., inhibiting Y. pestis, B. burgdorferi, or HIV infectivity) in a subject (e.g., a patient) in need of such treatment, comprising administering to the subject an effective amount of a bacterium of the invention, under conditions effective to elicit an immune response in the host to the polypeptide of interest, and thus to inhibit infection by, or pathogenic activity of, a pathogen carrying that polypeptide. Another aspect of the invention is a method for treating a patient infected by, or subject to infection by, a pathogen, or for preventing the spread of a pathogen from an infected patient to others, comprising administering to the patient an effective amount of a bacterium of the invention, under conditions effective for the bacterium to elicit an immune response which results in the inhibition of infectivity by, or a pathogenic activity of, a pathogen of interest.

The hosts (or targets) for administration of, and/or colonization by, the genetically altered bacteria include: uninfected individuals who are at risk for infection by the pathogen of interest; individuals already infected with the pathogen of interest; various animals infected by or subject to infection by a pathogen, e.g., a mammal, such as wildlife (e.g. a mouse, chipmunk, squirrel, shrew, vole, rat, raccoon, opossum, skunk, rabbit, deer, etc), an experimental animal, a farm animal (e.g. a cow, horse, etc.), pet (e.g., a dog or cat), or the like. In preferred embodiments, the animal is a primate, most preferably a human.

The immunogenic bacterium can be administered to a subject using any known technique that allows the bacterium to reach a suitable mucosum and to induce antibody formation and/or cell-mediated immunity. Typical mucosa-containing tissues, which are collectively referred to as mucosa-associated lymphoid tissue (MALT), include the gut-associated lymphoid tissue (GALT) and the bronchus-associated lymphoid tissue (BALT). Preferably, the bacterium is administered through the oral cavity, intranasally, or (e.g., in the case of an HIV vaccine) is applied directly to the rectum or the vagina, using conventional methods. Other suitable means of administration will be evident to the skilled worker. Delivery of engineered bacteria to a desired mucosal surface depends on the accessibility of the area and the local conditions. For example, engineered bacteria may be placed in a pharmaceutically acceptable solution, such as a saline solution, in a pill or capsule, or in a foam for delivery onto the vaginal or rectal mucosa.

In one embodiment, the bacteria are administered orally. For example, the bacteria can be administered by oral ingestion (e.g. in liquid solution, emulsion or the like, such as an elixir; or in a solid preparation, such as a tablet, caplet, capsule, pill, liquid-filled capsule, gelatin, or the like) or by gastric intubation. When administered in a liquid, the bacteria can be suspended, e.g., in a solution of about 0.16M sodium bicarbonate (pH about 8.5) to pre-gavage the subjects and to deliver the bacteria to neutralize the acidic environment of the stomach. Further suitable formulations for oral administration are discussed further below.

In another embodiment, the bacteria are administered intranasally, e.g., by broncho-nasal spraying or other forms of aerosol delivery. A nasal preparation can be liquid and can be administered, e.g., via aerosol, squeeze spray or pump spray disperser.

For delivery into the vaginal or rectal mucosa, foams can be used, which can include, e.g., one or more hydrophobically modified polysaccharides such as cellulosics and chitosans. Cellulosics include, for example, hydroxyethyl cellulose, hydroxypropyl cellulose, methyl cellulose, hydroxypropylmethyl cellulose, hydroxyethyl methyl cellulose, and the like. Chitosans include, for example, the following chitosan salts; chitosan lactate, chitosan salicylate, chitosan pyrrolidone carboxylate, chitosan itaconate, chitosan niacinate, chitosan formate, chitosan acetate, chitosan gallate, chitosan glutamate, chitosan maleate, chitosan aspartate, chitosan glycolate and quaternary amine substituted chitosan and salts thereof, and the like. Foam can also include other components such as water, ethyl alcohol, isopropyl alcohol, glycerin, glycerol, propylene glycol, and sorbitol. Spermicides are optionally included in the bacterial composition. Further examples of foams and foam delivery vehicles are described in, e.g., U.S. Pat. Nos. 5,595,980 and 4,922,928.

Alternatively, the bacteria can be delivered as a suppository or pessary. See, e.g., U.S. Pat. No. 4,322,399. In some embodiments, the bacteria of the invention are prepared in a preservation matrix such as described in U.S. Pat. No. 6,468,526 and are delivered in a dissolvable element made of dissolvable polymer material and/or complex carbohydrate material selected for dissolving properties, such that it remains in substantially solid form before use, and dissolves due to human body temperatures and moisture during use to release the agent material in a desired timed release and dosage. See, e.g., U.S. Pat. No. 5,529,782. The bacteria can also be delivered in vaginal foam or a sponge delivery vehicle such as described in U.S. Pat. No. 4,693,705.

In one embodiment, a bacterium of the invention is administered to a subject by coating, at least in part, a biologically compatible prosthetic device or dildo-like device with the bacterium, and then inserting the coated device into the subject. The biologically compatible device may comprise polymers such as fluorinated ethylene propylene, sulfonated polystyrene, polystyrene, or polyethylene terephthalate, or glass. The device may be, e.g., a catheter such as a urinary or peritoneal catheter, an IUD, or another intravaginal, intrauterine, or intraurethral device. In another embodiment, the device is a condom. In another embodiment, the device is a dildo-like device (e.g., a dildo comprising a small camera at one end, which allows one to follow the administration of the substance). Alternatively, if desired, the device (e.g., a relatively long-term device, such as an IUD) can be coated in vivo by administering the bacterium prior to insertion of the device, and allowing an indigenous protective flora to be formed on the device.

The particular mode of administration and the dosage regimen will be selected by the attending clinician, taking into account the particulars of the case (e.g., the subject, the degree of the infection, etc.). Treatment may involve yearly, monthly, daily or multi-daily doses, over a period of a few days to months, or even years. Even less frequent treatments can be used if the bacterium remains stably associated with the mucosa and continues to elicit an immune response for an extended period of time.

Optionally, antibiotic pretreatment of the subject can be used to pre-clear the mucosal surface of resident bacteria prior to introduction of the bacteria of the invention into the subject. See, e.g., Freter et al. (1983) Infect. Immun. 39, 686-703. Antibiotics can be provided orally or can be applied directly, e.g. to the vagina or rectum.

Certain agents that do not irritate mucosal epithelial cells may also be added to a unit dose of the bacteria in capsules or tablets to aid in colonization. Many bacteria on mucosal surfaces secrete capsular materials that coalesce to form a biofilm that covers the entire mucosal surface. It may be beneficial to add an enzyme that digests this biofilm material to promote penetration of the engineered bacteria into the biofilm for more successful colonization. The enzymes include DNAses, peptidases, collagenases, hyaluronidases, and other carbohydrate degrading enzymes. Antibiotics to which the engineered bacteria itself is not susceptible may also be added to decrease the number of resident bacteria on the mucosal surface in order to make room for the engineered bacteria.

The dosage form of a pharmaceutical composition will be determined by the mode of administration chosen. For example, topical and oral formulations can be employed. Topical preparations can include creams, ointments, sprays and the like. Oral formulations may be liquid (e.g., syrups, solutions or suspensions), or solid (e.g., powders, pills, tablets, or capsules). Sprays or drops, such as oral sprays or drops, are also included. For solid compositions (e.g., lyophilized bacteria), conventional non-toxic solid carriers can include pharmaceutical grades of mannitol, lactose, starch, or magnesium stearate. In a preferred application, live bacteria are lyophilized and placed in an enteric-coated capsule, which allows the bacteria not to be released until they have passed through the acidic stomach and reached the more alkaline colon. Actual methods of preparing suitable dosage forms are known, or will be apparent, to those skilled in the art.

The exact amount (effective dose) of the immunogenic bacteria can vary from subject to subject, depending on, i.e., the species, age, weight and general or clinical condition of the subject, the severity or mechanism of any disorder being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents. In general, normal dosage amounts may vary from about 10⁸ bacteria to 10¹¹ bacteria for humans and large animals, such as pigs, per 1 to 30 days. For example, a daily dose of about 10⁹ to 10¹⁰ E. coli Nissle 1917 is typically used for therapy of gastrointestinal complaints. A dose of 108 lactobacilli can be used to restore the normal urogenital flora. See, e.g., Reid et al. (2001) FEMS Immuno. Med. Microbiol. 32, 37-41. For small animals such as mice, a daily dose of about 10⁹ to 10¹⁰ bacteria is suitable.

The dosage may be varied by administering the bacteria by various regimens. For example, for experimental studies with mice, the bacteria can be administered once every 2-4 weeks (in total 2-4 times; first priming and subsequently one to three boosts. Alternatively, the bacteria (e.g. Lactobacilli) can be administered on two or three consecutive days, and this process repeated after two to four weeks, either once or two or three times.) Another dosage regimen is to administer 10⁸-10⁹ E. coli, or 2×10⁹ Lactobacillus, on days 1, 2, 3, 4, 8, 9, 10 and 11, followed by additional boosts of 4 days each, which brings the total number of inoculations to 20. This differs from some conventional procedures, in which only 5 inoculations are performed. In another dosage regimen, mice are boosted in intervals of 15 days rather than 10 days.

In some embodiments, applications of engineered bacteria to a mucosal surface will need to be repeated on a regular basis; optimal dosing intervals are routine to determine, but will vary with different mucosal environments and bacterial strains. The dosing intervals can vary, e.g., from once or twice daily to once every 2-4 weeks, to once a year. An advantage of using Lactobacillus is that, unlike many other bacteria, such as mycoplasma, a Lactobacillus bacterium can be administered multiple times, and/or at high dosages, without evoking toxicity or tolerance. In one embodiment, the bacteria need be delivered very infrequently (e.g., only once).

A bacterium of the invention can be formulated as pharmaceutical composition, which comprises the bacterium (e.g., a therapeutically effective amount of the bacterium) and a pharmaceutically acceptable carrier, using conventional components and methodologies. “Therapeutic” compositions and compositions in a “therapeutically effective amount” are compositions that can elicit at least a detectable amount of inhibition or amelioration of infection by, or a pathogenic activity of, a pathogen.

Such pharmaceutical compositions are normally formulated with a solid or liquid carrier, depending upon the particular mode of administration chosen. The pharmaceutically acceptable carriers useful in this disclosure are conventional. See, e.g., Remington, The Science and Practice of Pharmacy, current edition, for a discussion of pharmaceutically acceptable carriers. Pharmaceutically and physiologically acceptable fluid vehicles, such as water, physiological saline, other balanced salt solutions, aqueous dextrose, glycerol or the like, may be employed. Excipients that can be included are, for instance, other proteins, such as human serum albumin or plasma preparations. If desired, the pharmaceutical composition to be administered may also contain minor amounts of non-toxic auxiliary substances, such as wetting or emulsifying agents, preservatives, and pH buffering agents and the like, for example sodium acetate or sorbitan monolaurate. Gelatin capsules can serve as carriers for lyophilized vaccines.

Conventional adjuvants may also be added to enhance antigenicity, if desired. However, the antigenic bacteria of the invention can, themselves, serve as adjuvants.

The present disclosure also includes combinations of agents of the bacteria of the invention with one another, and/or with one or more other agents useful in the treatment of a pathogenic infection. For example, bacteria of the invention may be administered in combination with effective doses of conventional anti-pathogenic agents for treatment of Y. pestis infection, such as aminoglycosides (e.g., streptomycin or gentamicin), tetracyclines or floroquinalones (e.g., doxyxycline or cipro). Combinations with one or more antiretroviral drugs can be used for treatment of HIV infection. Combinations with doxycycline may be used for treatment of Lyme disease (Borrelia). The term “administration in combination” refers to both concurrent and sequential administration of the active agents. The combination therapies are of course not limited to the agents provided herein, but include any composition for the treatment of pathological infections.

Another aspect of the invention is a kit for carrying out any of the methods of the invention. For example, one embodiment is a kit for inhibiting an infection by a pathogen, comprising an effective amount of amount of an immunogenic bacterium of the invention and, optionally, (e.g., if the infection is in a subject in vivo) means for storing or packaging the bacterium, or for administering it to a subject.

The components of the kit will vary according to which method is being performed. Optionally, the kits comprise instructions for performing the method, e.g. instructions defining a vaccine regimen. Other optional elements of a kit of the invention include suitable buffers, media components, or the like; containers; or packaging materials. The reagents of the kit may be in containers in which the reagents are stable, e.g., in lyophilized form or stabilized liquids. The reagents may also be in single use form, e.g., in single dosage form for use as vaccines.

The present inventors have generated Lactobacilli which express a mutant Borrelia burdorferi OspA, in which the Cysteine (C) residue at position 17 has been modified to an Aspartic acid (D). The amino acid sequence of this modified leader sequence in the OspA protein is MKKYLLGIGLILALIAD (SEQ ID NO:3); this mutant leader is sometimes referred to herein as LipA_{c17d}, or, generically, as a “Cys-17” mutation. In the term “c17d,” c17d stands for the mutation of Cys-17 to aspartic acid (D). The mutation from cysteine to aspartic acid is also sometimes referred to herein as “c17D.” The mutated leader sequence can be encoded by any suitable nucleic acid, e.g. a nucleic acid having the sequence ttg gga ata ggt cta ata tta gcc tta ata gca gat (SEQ ID NO:4). This mutant leader lacks a suitable Cys (at residue 17 of the wild type sequence) that can be lipidated.

The inventors have found that this mutated OspA, when expressed in Lactobacilli, appears to be localized in the membrane, but not on the outer surface, of the bacterium. Surprisingly, Lactobacilli expressing the mutant protein elicit immune responses that are at least as strong as those elicited by Lactobacilli expressing wild type OspA. Therefore, such bacteria can serve as immunogenic compositions, such as vaccines, against Borrelia infection.

Furthermore, the fusion of such a mutant leader sequence to a polypeptide, such as detectable polypeptide, can be used to direct the polypeptide to the membrane. Such constructs can thus be useful experimentally, e.g., to determine details of the mechanism by which the proteins are transported to the membrane, or to isolate agents that enhance or inhibit the transport.

One aspect of the invention is a Lactobacillus bacterium that expresses a variant OspA polypeptide from Borrelia burgdorferi, wherein the cysteine at position 17 is replaced with another amino acid (e.g. an amino acid that cannot be lipidated, such as an aspartic acid); and/or that comprises an expressible polynucleotide encoding a variant OspA polypeptide from Borrelia burgdorferi, wherein the cysteine at position 17 is replaced with another amino acid (e.g. an amino acid that cannot be lipidated, such as an aspartic acid), and, optionally, wherein, the 5′ terminal sequence encoding the leader is modified so that the codon usage is optimized for expression in Lactobacillus. If desired, to remove a putative autoreactive epitope, amino acids 165-173 of the OspA can be replaced with a sequence from a less arthritogenic species (e.g., B. afzelii, strain PGau), as is discussed in Example V.

In the foregoing and in the following examples, all temperatures are set forth in uncorrected degrees Celsius; and, unless otherwise indicated, all parts and percentages are by weight.

### EXAMPLES

### Example I

### Materials and Methods

### A. Clones Available in Our Laboratory:

TUNER(DE3)pET28a-LcrV (Kan+, Cm+): this clone contains Y. pestis LcrV cloned in an E. coli expression vector. Protein expression is induced by IPTG.

TUNER(DE3)pET28a-YopD (Kan+, Cm+): this clone contains Y. Pestis YopD cloned in an E. coli expression vector. Protein expression is induced by IPTG.

AAEC185pCAF1, tet+: this clone has Y. pestis F1 cloned in an E. coli regular vector. F1 expression in induced by shifting cultures from 28 C to 37 C overnight.

### B. Clones to be Used for Challenge Studies:

Y. pestis, strain Colorado 92 (C092), F1+

Y. pestis, F1

### C. Purification of Yersinia Proteins:

Recombinant LcrV and YopD: Escherichia coli (strain BL21 (DE3)) is transformed with the plasmid encoding the target gene, grown in 1000 mL TBY media (5 g/l NaCl, 10 g/l tryptone, 5 g/l yeast extract, 25 μg/l chloramphenicol and 30 μg/l kanamycin) at 37° C. in a shaking incubator. When the OD₆₀₀ reaches 0.4-0.6 units, protein expression is induced by adding IPTG (isopropyl B-D-thiogalactopyranoside) to a final concentration of 1.0 mM and cells are grown for another 3 h. The cultures are harvested by centrifugation in 160 mL volumes at 5000×g, 5 min, at 4° C. The cell pellets are stored at −20° C. until use. Once thawed, the cell pellets are incubated at room temperature with 3.2 mL CelLytic B-II (Sigma) and DNase I (5.0 μg/mL) for 15 min. Cellular debris is pelleted by centrifugation at 25000×g for 15 min at 4° C. and the combined supernatant “crude extract” is used for further purification of the recombinant protein. The crude extract is applied directly to a chromatography column containing 75 mL His-Select HC Nickel Affinity Gel (Sigma) previously equilibrated with buffer (50 mM NaPO₄ pH8.0 and 0.3M NaCl) at a flow rate of about 1.0 mL/min. The column is then washed with additional volumes of equilibration buffer until the A₂₈₀ of the material eluting from the column is stable and near that of the equilibration buffer. The recombinant protein is eluted off the column in approximately 80 mL of elution buffer (50 mM NaPO₄ pH8.0, 0.3M NaCl and 250 mM Imidazole) and then concentrated to 10 mL in a ultrafiltration cell using a molecular cutoff weight of 30 Kd for LcrV (37 Kd) and YopD (45 Kda) (Centriprep, Millipore). The protein concentration is determined by the Bradford protein assay (Biorad) and stored at −70° C.

D. Recombinant F1. This protocol has been adapted from Miller et al. (1998) FEMS Immunol Med Microbiol 21, 213-21. All buffers contain 0.5M PMSF (Sigma) and are filtered before use. Water used in chromatographic procedures is Mili-Q purified. E. coli transformed with the F1 expression vector is cultured in 3 L media at 37° C. for 19 h. The cultures are centrifuged at 14,000×g for 45 min at 4° C. and the cell pellet and flocculant layer resuspended in 200 ml phosphate-buffered saline (PBS), pH7.2 and incubated at room temperature for 30 min. The suspension is centrifuged at 14,000×g for 30 min and the supernatant is added to 50% ammonium sulfate saturation. After stirring for 1 h, the fractionate is centrifuged for 14,000×g for 30 min at 4° C. The ammonium sulfate pellet is resuspended in PBS and dialyzed against 4×2 L of the same buffer, overnight. The dialyzed crude extract is centrifuged at 27,000×g to remove insoluble material, followed by filter sterilization (0.22 um Amicon). Aliquotes of 200 ul of the dialyzed extract are applied to an FPLC Superose 12HR 10/30 column previously calibrated with PBS. The rF1 is eluted with the same buffer at a flow rate of 0.5 ml/min. The peak fraction is collected and analyzed for rF1 by SDS-PAGE and Western Blot. Three unlipidated full length proteins, rLcrV, rF1, and rYopD, are cloned and purified. These are used as positive controls to compare to the immune responses generated against the oral Lactobacillus vaccine candidates.

### E. Immunological Methods Used to Monitor Immune Responses to the Vaccine Antigen:

Cell culture: Spleens or other lymphoid tissues are teased apart, and single cell suspensions are centrifuged on Ficoll-Hypaque (SG 1.083). When necessary, erythrocytes are removed by lysis in buffered ammonium chloride. Cells are finally resuspended in RPMI 1640 medium plus 10% fetal calf serum, and counted in a hemacytometer with Trypan blue to assess viability. For culture, the medium is supplemented with antibiotics, non-essential amino acids, glutamine, sodium pyruvate, and HEPES buffer.

ELISPOT assay to enumerate antibody-secreting B cells: The protocol suggested by the manufacturer of ELISPOT assay (Millipore Corp., Bedford Mass.) is followed. Cells are enumerated using membrane-based 96-well plates coated with antigen or isotype-specific anti-Ig as for ELISA, and blocked. Cell suspensions (10³-2×10⁵ per well) are incubated for 3 h at 37° C. After washing, the plates are developed with peroxidase-conjugated anti-Ig reagents as for ELISA, but using 3-amino-9-ethylcarbazole/H₂O₂ as substrate to forms spots of color on the membrane. Spots are counted under a low power stereo microscope, and related to the number of cells plated. Samples are tested in duplicate using at least 2 different cell concentrations.

ELISPOT assay to enumerate the IL-4- and IFNgamma-secreting T cells: The protocol suggested by the manufacturer of ELISPOT kits (R&D systems) is followed. Briefly, the splenocytes are incubated in anti-IL-4 and anti-IFNgamma antibody-coated ELISPOT plates, varying the number of cells from immunized mice to achieve a final cell density of 5×10⁵ per well using feeder cells from unimmunized mice. The cultures are established in complete RPM11640 medium with or without specific recombinant Y. pestis antigen as stimulant. The cultures are incubated for 48 hours at 37° C. in 5% CO2. Then the cells are lysed with water, the plates are washed, and incubated with the biotinylated secondary antibody followed by avidin-peroxidase conjugate. Spots are developed using appropriate substrate and counted using a colony counting microscope.

Quantification of IL-4 and IFNgamma secreted by immune spleen cells: Single-cell suspensions are prepared from spleens of the vaccinated and control inoculated mice. The red blood cells are lysed with ACK solution (150 mM NH4CI, 1 mM KHCO3, 0.1 mM Na2EDTA, pH 7.3). The splenocytes are cultured in 96-well plates at a concentration of 5×10⁵ cells/well in presence of specific antigens [1-5 ug of recombinant Y. pestis proteins, and various doses of vaccine antigen, 0.5 ug of concanavalin A (conA), or no additives (unstimulated control)]. Complete RPM11640 media is used for culturing the cells. After 4 days, the culture supernatants are collected and tested for the presence of IL-4 and IFNgamma using commercially available antigen-capture ELISAs (R&D Systems). All assays are performed in triplicate and the amount of cytokines in the culture supernatants is quantified using a linear regression equation obtained from the optical density values of the IL-4 and IFNgamma standards.

### Example II

### Construction and Testing of Lactobacillus plantarum (Strain 256) Bacteria which Express Polypeptides that Contain, at their N-Termini an OspA Leader Sequence (Either Wild Type or Modified)

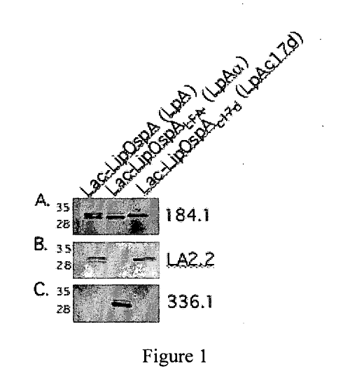

Lactobacillus plantarum (strain 256) was transformed individually with three DNA constructs, as indicated in FIG. 1. A description of the DNA constructs used to make these bacteria is presented below. Protein extract was obtained from each of the induced bacteria and was subjected to Western blot analysis against anti-OspA monoclonal antibodies 184.1, LA2.2 and 336.1. The specificities of the monoclonal antibodies are as follows: mAb 184.1 is specific for the N-terminal region of B. burgdorferi OspA; mAb LA2.2 is specific for the OspA epitope from amino acids 165-173; mAb 336.1 is specific for the C-terminal region of B. afzelii. The antigenicity of each of the bacteria (vaccines), as determined by immunoblotting with the three monoclonal antibodies against OspA, is shown in FIG. 1.

Lac-LipOspA, or LpA: The DNA construct used to make this bacterium contains full-length OspA, in which the OspA leader sequence is N-terminal to the remaining coding sequences of the protein.

Lac-LipOspA_{c17d} or LpAc17d: The DNA construct used to make this bacterium contains full-length OspA, in which the OspA leader sequence is N-terminal to the remaining coding sequences of the protein, and the Cys residue at position 17 is substituted with an aspartic acid.

Lac-LipOspA_{LFA}- or LpAα: The DNA construct used to make this bacterium contains full-length OspA, in which the OspA leader sequence is N-terminal to the remaining coding sequences of the protein, and a putative autoreactive epitope, amino acids 165-173, is replaced with a sequence from a less arthritogenic species (B. afzelii, strain PGau). The following procedure was used to generate the OspA construct lacking the epitope cross-reactive with hLFA1. To mutate the sequence 165-173 on the β-13 region of OspA from B. burgdorferi, we had to consider the maintenance of charge parity. Changing T170 to K would require changing V179 to E on the next β strand. To address both issues we replaced the residues 161 to 190 in OspA from B. burgdorferi B31 with the analogous region from a non-arthritogenic European species, B. afzelii, that had these compensation changes. To further stabilize the C-terminus of the mutant OspA molecule, which is important for inducing a protective immune response, we needed to replace it with the analogous C-terminus sequence from the same genospecies used to knock out the hLFA1 cross-reactive epitope. This construct, containing OspAB31₁₋₁₆₀PGau₁₆₁₋₁₉₀B31₁₉₁₋₂₁₅Pko₂₁₆₋₂₇₃, is referred to as LipOspA_{LFA}.

The sequence of the nucleic acid encoding LpAα is:

(SEQ ID NO: 5)

ATGAAAAAATATTTATTGGGAATAGGTCTAATATTAGCCTTAATAGCATG
TAAGCAAAATGTTAGCAGCCTTGACGAGAAAAACAGCGTTTCAGTAGATT
TGCCTGGTGAAATGAAAGTTCTTGTAAGCAAAGAAAAAAACAAAGACGGC
AAGTACGATCTAATTGCAACAGTAGACAAGCTTGAGCTTAAAGGAACTTC
TGATAAAAACAATGGATCTGGAGTACTTGAAGGCGTAAAAGCTGACAAAA
GTAAAGTAAAATTAACAATTTCTGACGATCTAGGTCAAACCACACTTGAA
GTTTTCAAAGAAGATGGCAAAACACTAGTATCAAAAAAAGTAACTTCCAA
AGACAAGTCATCAACAGAAGAAAAATTCAATGAAAAAGGTGAAGTATCTG
AAAAAATAATAACAAGAGCAGACGGAACCAGACTTGAATACACAGGAATT
AAAAGCGATGGATCTGGAAAAGCTAAAGAGGTTTTAAAAGGCTTTACTCT
TGAAGGAAAAGTAGCTAATGATAAAGTAACATTGGAAGTAAAAGAAGGAA
CCGTTACTTTAAGTAAGAATATTTCAAAATCTGGGGAAGTTTCAGTTGAA
CTTAATGACACTGACAGTAGTGCTGCTACTAAAAAAACTGCAGCTTGGAA
TTCAAAAACTTCTACTTTAACAATTAGTGTTAACAGCAAAAAAACTACAC
AACTTGTGTTTACTAAACAAGACACAATAACTGTACAAAAATACGACTCC
GCAGGTACCAATTTAGAAGGCACAGCAGTCGAAATTAAAACACTTGATGA
ACTTAAAAACGCTTTAAAATAA

Results: The recombinant protein Lac-LipOspA (LpA) shares an antigenic determinant with the mutants Lac-LipOspA_{c17d} (LpAc17d) and Lac-LipOspA_{LFA}-(LpAα); all bind to the mAb 184.1. The mutant lacking the epitope cross-reactive with hLFA, Lac-LipOspA_{LFA}-(LpAα), has the mAb LA2.2 binding site disrupted and therefore, as expected, does not bind this mAb. The monoclonal antibody 336.1 is not expected to bind to LpA and LpAc17d, but binds to LpAα, as expected, because it binds to B. afzelii strains rather than B. burdgorferi.

LpAβ: We will modify the LpAα construct by substituting the Cys residue at position 17 with an aspartic acid and will transform the modified construct into Lactobacillus plantarum (strain 256). The antigenicity of this vaccine candidate will be tested by immunoblot as above, in comparison with the parental construct (LpAα). It is expected to be at least as good as that of the parent strain.

We will also further modify each of the constructs noted above, so that some or all of the “ata” codons in the leader sequence are mutated to either “atc” or “att”, to optimize the codon usage for expression in Lactobacillus.

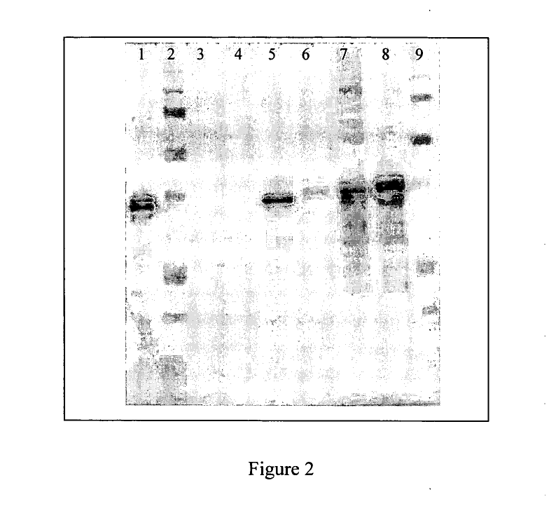

We also cloned LcrV (serotype O:3) into a Lactobacillus plantarum expression vector using a Borrelia burgdorferi OspA leader sequence (Lac-LipALcrV) and confirmed expression of the antigen (FIG. 2).

### Example III

Oral immunization of mice with Lactobacillus expressing an antigen of interest (the B. burgdorferi OspA antigen) and determination of the systemic antibody response to the immunizing antigen. An oral vaccine against Lyme disease for human use based in Lactobacillus expressing B. burgdorferi's OspA.

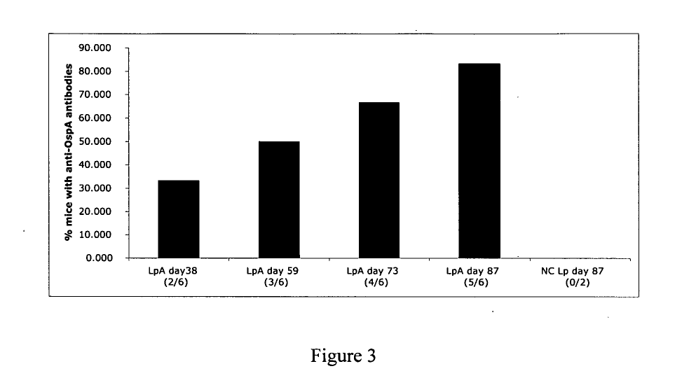

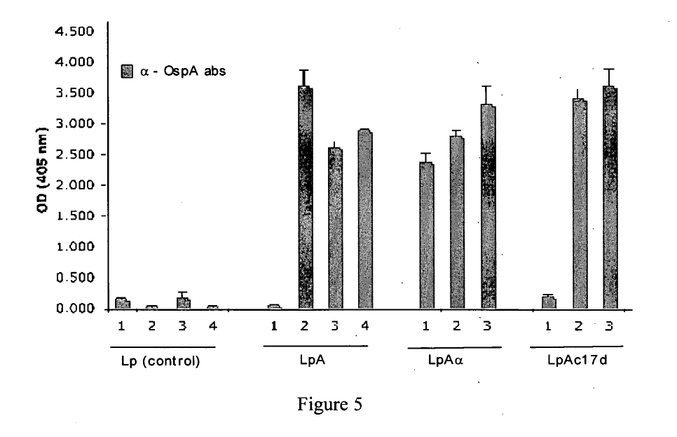

We tested L. plantarum expressing OspA (LpA) by oral gavage inoculation in C3H-HeN mice in comparison with a control comprised of the parental strain (L. plantarum (Lp)). We checked the systemic IgG response to the OspA antigen and verified that 2/6 mice developed antibodies to OspA after the immunization (day 38); 3/6 mice developed antibodies after the 1^{st} boost (day 59), then one more mouse (4/6, 66%) developed antibodies after the second boost (day 73); and one more developed Abs to OspA after a third boost (day 87). Overall, 5/6 (83%) mice developed antibodies against OspA, where we observed an increase of the total level of antibody production with time (FIG. 3). In another experiment, in which we optimized the vaccine dose, we verified that 3/4 mice (75%) developed antibodies against OspA after the second boost (day 70) (FIG. 5).

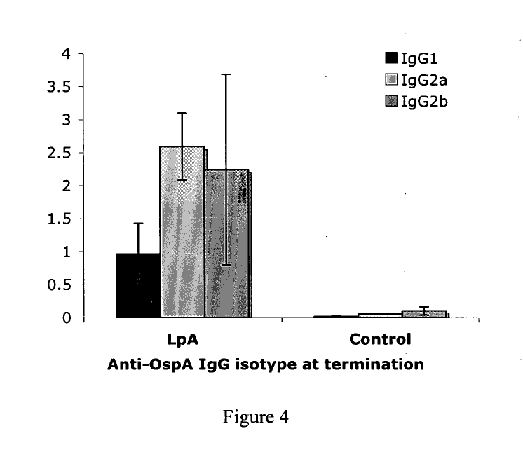

Furthermore, we determined the IgG subclass induced by oral immunization with L. plantarum expressing OspA (LpA) in comparison with the control (Lp) by ELISA (FIG. 4). Oral immunization with L. plantarum expressing OspA, resulted in an OspA-specific IgG subclass distribution skewed toward IgG2a/2b and IgG1 was the lowest subclass detected.

These results indicate that the oral administration of an L. plantarum based vaccine elicited a sustainable anti-OspA systemic immune response (IgG) in mouse serum for over three months and that immune response was skewed towards IgG2a/2b production.

### Example IV

### A Modified OspA Leader Sequence which is Mutated at the Cys-17 Position

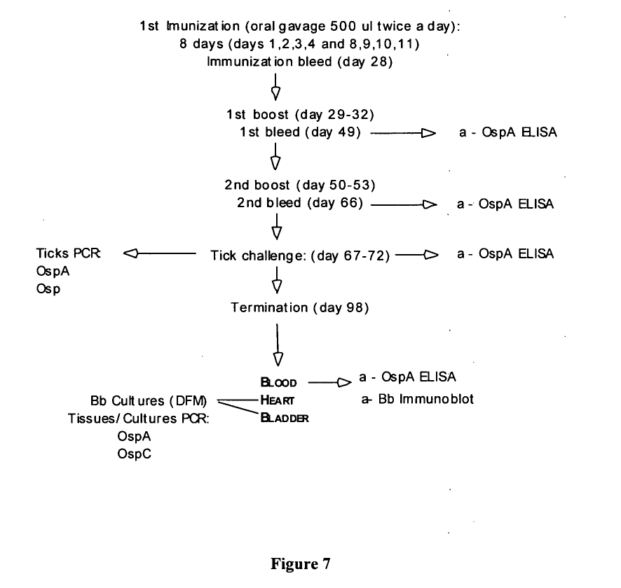

We mutated OspA at amino acid 17 (cysteine to aspartic acid), which would be expected to knock out the lipidation cleavage site. The mutant is referred to as LpAc17d. (See Example II for a further discussion of this and the other constructs used in the present Example.) We have obtained evidence suggesting that in this mutant, OspA is not expressed at the surface of the Lactobacillus strain. Three different constructs of B. burgdorferi OspA [LpA (having a wild type OspA leader); LpAc17d; and LpAα] were transformed into L. plantarum, and inoculated by oral gavage into C3H-HeN mice: LpA, 4 mice; LpAα, 3 mice; LpAc17d, 3 mice; and the negative control was the parental Lactobacillus strain (Lp, 4 mice). The mice were subjected to the protocol shown in FIG. 7. OspA ELISA assays were performed on sera taken two days before challenge with B. burgdorferi infected field ticks (day 66, see FIG. 7); the OD values are shown in FIG. 5. The results indicated that mutants of OspA such as LpAc17d can induce an equivalent anti-OspA immune response compared to the wild type OspA (LpA) after the last immunization. Immunized mice that developed a high IgG response to the oral vaccine are expected to be protected from infection.

It therefore appears that OspA comprising this mutant OspA leader, and expressed in at least this strain of Lactobacillus, is at least as immunogenic as wild type OspA.

### Example V

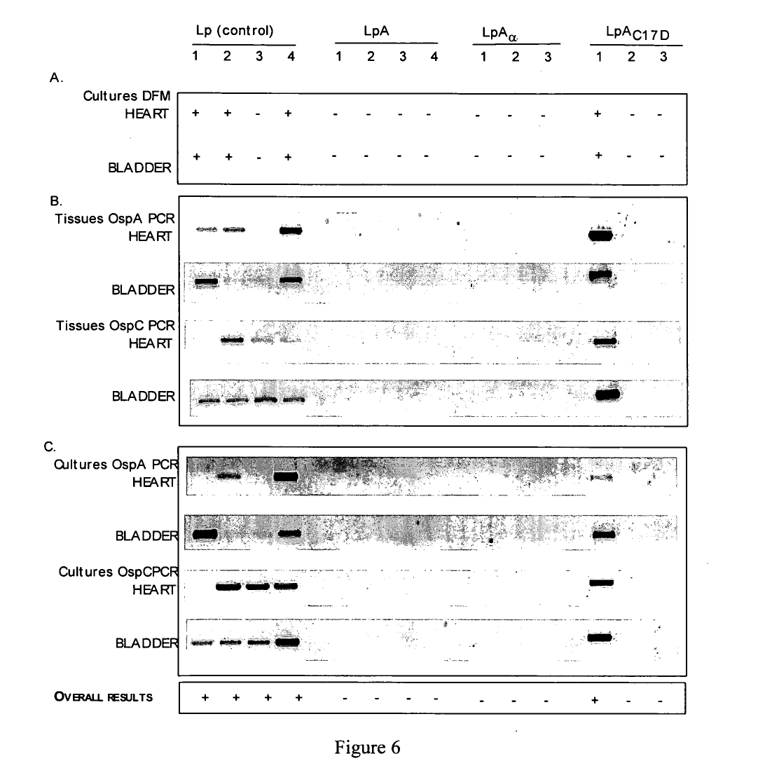

Mice vaccinated orally as described in Example IV (with Lactobacillus bacteria expressing LpA, LpAc17d, or LpAα) were analyzed with regard to expression of B. burgdorferi OspA or OspC in various tissues following vaccination with strains of the invention and challenge by B. burgdorferi. We compared the heart and bladder culture results of tissues harvested one month after challenge (FIGS. 6A, 6B and 6C). Culture results were determined by dark field microscopy confirmed by PCR (OspA, 820 bp and OspC, ˜600 bp). PCR was performed in blinded samples of the culture and tissue independently. Result: Borrelia burgdorferi does not disseminate in 100% of C3H-HeN mice vaccinated orally with LpA and LpAα and in 67% of mice vaccinated with LpAc17d.

### Example VI

### Challenge of Immunized Mice and Determination of Vaccine Efficacy

A. An E. coli Based Vaccine Carrier

We determined if the systemic anti-OspA immune response elicited by our oral vaccine could protect mice from Bb infection in vivo. After immunization, mice were challenged by infestation with I. scapularis nymphs carrying an array of Bb strains. Infection or protection was determined by the presence or absence of Bb dissemination, respectively. Field ticks represent a more authentic challenge to the immunized mice than needle inoculation because they harbor an array of infectious and non-infectious strains of Bb that the vertebrate host of the spirochete would be exposed to in the wild. One month after challenge, spirochete dissemination in vaccinated and control mice was detected by immunoblotting serum against whole cell sonicate of Bb, by culture of Bb from infected heart and bladder tissues and by PCR amplification of Bb ospC DNA from cultures and from heart, bladder and ear tissues. We concluded that 89% of mice vaccinated orally with E. coli expressing OspA were protected from Bb dissemination after tick challenge. All negative controls had evidence of Bb dissemination and therefore were not protected.

B. A Lactobacillus Based Vaccine Carrier

We followed the experimental design previously developed for E. coli and we concluded that OspA antibody response results in protection from Bb infection in 100% of the mice vaccinated orally with LpA, 100% of the mice vaccinated with LpAα and 67% of the mice vaccinated with LpAc17d, while all controls were infected.

TABLE 1
Efficacy of Lactobacillus plantarum based vaccines

| | Serology | | | | | |
|---|---|---|---|---|---|---|
| | OspA- | Bb | | | | Tick |
| | ELISA | ImmunoBlot | Culture of Tissues | | Vertebrate | Vector % |
| | 2 d before | 1 mo post | Heart | Bladder | Host % | Bb |
| | challenge | challenge | DFM:PCR | DFM:PCR | Protection | Clearance |
| Lp | 0P/4 | 4P/4 | 4P/4 | 4P/4 | 0% | 4P/4 |
| (Control) | (0%) | (100%) | (100%) | (100%) | | (100%) |
| Lac-LipOspA | 3P/4 | 1E/4 | 0P/4 | 0P/4 | 100% | 0P/4 |
| (LpA) | (75%) | (0-25%) | (0%) | (0%) | | (0%) |
| Lac- | 3P/3 | 0P/3 | 0P/3 | 0P/3 | 100% | 2P/3 |
| LipOspABPBPk | (100%) | (0%) | (0%) | (0%) | | (67%) |
| (LpAα) | | | | | | |
| Lac- | 2P/3 | 1P/3 | 1P/3 | 1P/3 | 67% | 3P/3 |
| LipOspA_{C17D} | (67%) | (33%) | (33%) | (33%) | | (100%) |
| (LpA_{C17D}) | | | | | | |

P, Positive;
N, Negative;
E, equivocal;
DFM, Dark Field Microscopy

### Example VII

### Construction of the Y. pestis Antigens, LcrV (e.g., Serotype O:3), F1 and YopD in a Probiotic Vaccine Carrier, Lactobacillus Spp

The three Yersinia genes are cloned independently into a Lactobacillus expression vector downstream of a Borrelia burgdorferi OspA leader sequence and expression of the antigens is evaluated.

LcrV, F1 and YopD all lack a classical signal peptide at their N terminus. In these constructs, a B. burgdorferi's OspA signal peptidase II cleavage sequence that includes the Cys at position 17 is cloned at the amino terminus of a Y. pestis protein (LcrV, F1 or YopD). Without wishing to be bound by any particular mechanism, it is suggested that this sequence may provide a “transgenic” site for lipid modification (Pam3Cys) of these proteins.

For the generation of prototype vaccine candidates, the different antigens are cloned in expression systems that have been developed specifically for Lactobacillus species (see, e.g., Pouwels et al. (2001) Methods Enzymol 336, 369-89; J. F. Seegers (2002), supra; Mercenier et al. (2000) Curr Issues Mol Biol 2, 17-25). These expression systems are based on plasmids that have a cassette like structure that allows easy exchange of functions such as promoter sequence, secretion signal and selection marker. To construct Yersinia lipoproteins, the three genes are ligated individually downstream of B. burgdorferi OspA leader sequence by PCR using splicing by overlap extension (SOE). The resulting DNA fragment are then cloned in a Lactobacillus expression vector (pLAC613) for intracellular expression. One of these clones (Lac-LipLcrVO:3) was discussed in Example II. It was shown that a good yield of lipidated LcrV was expressed from it in Lactobacillus plantarum (See FIG. 2). The clones obtained are sequenced by ABI's BigDye terminator method. The sequences are checked on Sequencher™ 4.1 against the respective published data. Expression of antigen is confirmed by SDS-PAGE followed by Coomassie blue staining and by Immunoblot. For Immunoblot we use either monoclonal antibodies against LcrV and F1 [mab19.3, (Gomes-Solecki et al. (2005) Clin Diagn Lab Immunol 12, 339-46) and/or mAbYBF19, QED Bioscience Inc] or mouse polyclonal antibody against YopD. Polyclonal antibody against YopD is produced as follows. Starting with YopD cloned in an E. coli expression vector, this protein is induced and purified as described in Example 1 and 10 ug is used to immunize mice on day 1, followed by two boosts with the same amount of protein, on day 15 and on day 30. Two weeks later mice are sacrificed and polyclonal antibody blood is collected via cardiac puncture. In addition, a recombinant chimera of F1 and LcrV is constructed to be used as a positive control for the immunization studies. This vaccine is delivered subcutaneously.

Several Y. pestis chimeric lipoproteins are generated. For example, three contain a 17 amino acid B. burgdorferi OspA leader sequence cloned upstream of a Y. pestis gene (LcrV, F1 or YopD); and three contain a mutated B. burgdorferi OspA leader sequence (Cys-17) cloned upstream of one of the three Y. pestis genes. We have cloned and expressed lipidated LcrV in Lactobacillus plantarum (see FIG. 2). In these constructs, the LcrV gene, which in its wild type form lacks a classical signal peptide that is characteristic of a lipoprotein, is operably linked to either the wild type OspA leader sequence noted above or to the Cys-17 mutated version of the leader. The clones noted above are then transformed into different Lactobacillus spp strains, because the expression of antigens and adjuvanticity of different host strains can vary. The effect in different strains of Lactobacillus is determined empirically, because immunogenicity is not necessarily directly related to expression level of antigen. For example, the expression level often is higher in L. casei than in L. plantarum, but this provokes low, if any, immune response. Among the strains that are tested are L. casei 393 and L. plantarum 256. Other strains that are tested include L. reuteri or L. johnsonii.

If a heterologous polypeptide has toxic effects, thereby inhibiting growth of the host strain, this can be overcome in a number of ways. For example, the gene of interest can be cloned downstream of a regulatable promoter. Thus expression is reduced in the absence of a proper inducer. Alternatively, only the major antigenic determinant of the total protein can be cloned, which will abolish the function of the antigen.

Some heterologous proteins may be highly susceptible to proteolytic degradation by endoproteases, which can be detrimental to the yield of antigen in the cell. Most proteolytic sites have been well defined and the gene of the antigen can be analyzed for the presence of such sites. If they are in a region that is exposed, the antigen will likely be degraded. One solution to this problem is to remove or alter putative proteolytic sites by genetic engineering.

### Example VIII

### Oral Immunization of Mice with Lactobacillus Expressing Y. pestis Antigens and Determination of their Mucosal and Systemic Antibody Responses

These experiments are used to determine the kinetics of the immune response to the vaccine candidates.

8 week old female BALB/c mice are immunized with individual vaccine candidates, chimeric lipoproteins (B. burgdorferi OspA leader—Y. pestis Ag) such as Lac-lipLcrV, Lac-lip(c17d)LcrV; Lac-lipF1, Lac-lip(c17d)F1; and Lac-lipYopD, Lac-lip(c17d) independently, as well as several combinations of two and three antigens. Controls include mice given the recombinant antigen (positive), as well as mice sham-immunized only with Lactobacillus (negative). The mucosal and systemic immune response to the respective Y. pestis antigens is monitored by ELISA.

Preparation of vaccine antigen: Lactobacillus plantarum, strain 256 (or another Lactobacillus strain that has proven to have higher immunogenicity) is transformed with a vector carrying Y. pestis antigens described above. Typically, 1 ml of cells at an OD₆₀₀=1.0 contains 2×10⁹ cells, corresponding to approximately 125 ug total cellular protein. A single inoculation requires about 2×10⁹ cells. Batches are stored at −80° C. and thawed on ice before use. From frozen stock, 10 ml of MRS (Difco) supplemented with 10 ug/ml chloramphenicol (Cm) is inoculated and incubated 16-20 h (overnight) at 30° C.; the ON culture is diluted 100-fold in 10 ml LCM, supplemented with 10 ug/ml Cm and incubated overnight at 30° C. The following morning the ON culture is diluted 50-fold in 200 ml pre-warmed LCM [6.25 g yeast extract, 5 ml Tween 20, 2.5 g bi-ammonium citrate, 1.25 g sodium acetate, 0.33 g magnesium sulphate, 12.5 g trypticase, 3.75 g tryptose, 0.25 g cysteine, 0.05 g manganese sulphate per liter, buffered with 0.5 M potassium phosphate buffer (pH 7.0). Media is supplemented with 0.5% lactose to final concentration and with chloramphenicol (10 ug/ml) before use] to induce protein expression, supplemented with 10 ug/ml chloramphenicol and incubated at 30° C. to an OD₆₀₀=1. Cells are harvested by centrifugation, washed twice with 20 ml PBS (or once with PBS and once with NaHCO₃), and resuspended in PBS/10% glycerol in a final volume of 2 ml. The cell suspension is frozen immediately in liquid nitrogen and stored at −80° C. until use. For immunization, we allow the contents of the frozen vials to thaw slowly while keeping the vials on ice.

Oral immunization: 8 wk old female BALB/c inbred mice (Charles River, Boston) are used. 500 ul of previously induced bacteria are inoculated, twice a day (total of 2×10⁹ inoculum/day), intragastrically (i.g.), with a ball tipped disposable feeding needle, using a previously titrated dose of the selected antigen. Controls include mice given the antigen alone, as well as mice that are sham-immunized. For comparison, groups of mice are immunized subcutaneously (s.c.) using the same antigen dose and schedule. Mice receive the first immunization daily on days 1, 2, 3, 4 and 8, 9, 10, 11. Two weeks later, blood is collected by retro-orbital bleeding (day 27) and on days 30, 31, 32, 33 receive the 1^{st} boost. On day 45, they are bled for the second time and on days 52, 53, 54, 55 receive the 2^{nd} boost. On day 64, mice are bled for the 3^{rd} time. On day 67, 68, 69, 70 they receive the 3^{rd} boost and two weeks later mice are bled to check the immune response. Three days later, on day 88, mice are challenged with virulent Y. pestis and two weeks later, mice are euthanized and tissues are harvested.

Antibody assays: To assess the systemic immune response, IgG, total IgG and IgG subclass isotyping (IgG1, IgG2a and IgG2b) are performed in immunized mouse sera using either alkaline phosphatase (1:1,600) (KPL) or horseradish peroxidase secondary antibody (1:50,000) (Bethyl, Montgomery, Tex.) by ELISA against each Y. pestis antigen, LcrV, F1 and YopD. To assess mucosal immune response, the secretions to be routinely obtained are saliva and vaginal wash (from female mice), since these can be collected from live animals repeatedly, and they are tested by IgA isotype-specific ELISA against each Y. pestis antigen, LcrV, F1 and YopD which are purified as described in Example I.

These assays provide an indication of which vaccine candidates induce the most potent and quickest immune response as determined by the Ig-isotype specific assays. The most promising candidates are selected for further experiments: immunization followed by Y. pestis challenge, as described in Example VIII. In further studies, the immunization schedule is optimized, in order to develop an oral vaccine which raises a protective immunity in a timely fashion.

If a lipA fusion lipoprotein(s) is poorly expressed in Lactobacillus or not transported into the membrane, this might result in a poor stimulation of the immune system. Expression levels can be influenced by several factors, not related to the promoter activity. One is the codon use of the gene. Each bacterial species has a certain codon bias. This means that for each specific amino acid there is a preferred codon usage. This translates in an abundance of specific tRNAs and reduced levels of other tRNAs in the cellular cytoplasm. Also, the secondary structure of the mRNA can influence expression levels. Both factors can be overcome by either selecting another host strain or, preferably, designing a new gene that has the same amino acid sequence, but has been optimized for codon bias of the used strain and has better folding characteristics. In other words, these problems can be overcome by engineering different segments of the antigen, especially avoiding strongly hydrophobic sequences, and selecting shorter sequences defining epitope segments. Such procedures are conventional. Different expression strains or even species of bacteria, and adjustments in growth conditions, are also factors that can be manipulated to obtain satisfactory expression.

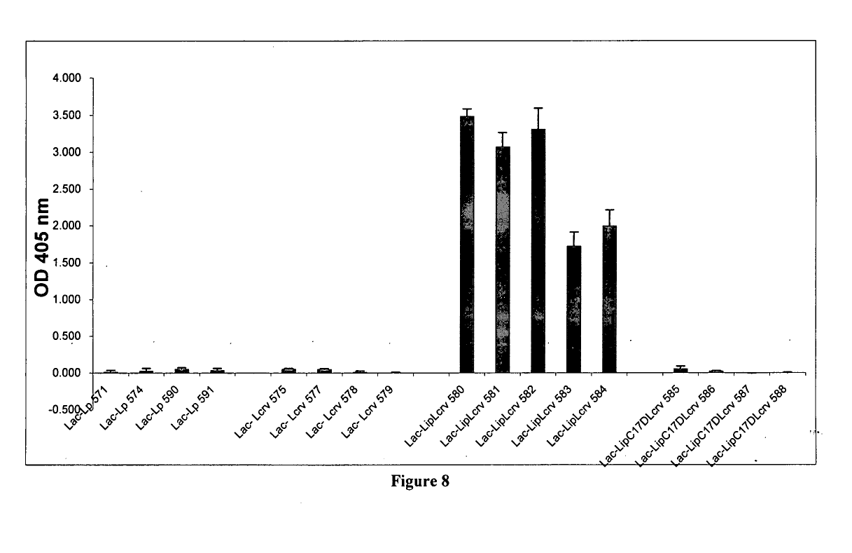

We prepared constructs in which LcrV was cloned downstream of the 17 amino acid B. burgdorferi OspA leader sequence or the Cys-17 mutant leader, or was cloned without any leader sequence, using methods as described in Example VII. These constructs were expressed in Lactobacillus plantarum strain 256 and introduced by oral immunization into mice, and ELISA assays were performed to assess systemic immunity, all as described above. The OD values of these ELISAs are shown in FIG. 8. These data clearly indicate that the Lactobacillus expressing the OspA leader/LcrV fusion polypeptide elicits a systemic immune response to LcrV after oral immunization, whereas the controls do not.

### Example IX

### Challenge of Immunized Mice with Virulent Y. pestis, to Determine Vaccine Efficacy

Each of the vaccine candidates shown to induce high systemic and mucosal response in Example VIII is tested in order to select the most promising candidates. Mice are challenged via aerosol inoculation with virulent Y. pestis, both F1+(strain C092) and F1. In future experiments, vaccine efficiency studies will be carried out using the immunogens selected as described herein, and challenge will be performed via aerosolized fully virulent Y. pestis.

Groups of ten mice are immunized orally with the recombinant Lactobacillus strains expressing chimeric lipoproteins (B. burgdorferi OspA leader fusion with Y. pestis Ag) such as, Lac-lipLcrV, Lac-lip(c17d)LcrV; Lac-lipF1, Lac-lip(c17d)F1; and Lac-lipYopD, Lac-lip(c17d) independently, as well as several combinations of antigens. Controls include mice given the recombinant antigen (positive), as well as mice sham-immunized only with Lactobacillus (negative). Samples of serum and secretions (see below) are collected 15 days after each vaccine dose and at termination to analyze for antibodies. Blood is collected by retro-orbital bleeding. Antigen-specific immune responses induced by each antigen are analyzed to determine if overexpression of the antigen leads to elicitation of increased levels of antibody and T cell responses. After immunization the mice that show a positive immune response are challenged via aerosol and via intranasal with Y. pestis F1 positive (strain C092) and an F1 negative strain. An additional strain is used to perform intranasal challenges, Y. pestis C092-pgm negative. Initial challenges are based upon the previously published LD50 data in Balb/c mice (2.3×10⁴ cfu for aerosol and 10⁴ cfu for intranasal) for the CO92 strain of Y. pestis. For aerosol challenge, mice are exposed to a small particle aerosol within a class III biological safety cabinet. The efficiency of infections is determined with several test mice within each group and a calculated efficiency of infection is determined. Survival and tissue burden in lungs are determined for each group (10 animals) of mice. For intranasal challenge, mice are anesthetized with isofluorane and inoculated with 50 ul of bacterial suspension (10⁴ cfu) via the nares with a fine pipette tip. The animals are monitored for two weeks. The symptoms of plague infection in general appear 3 days after inoculation: starry or ruffled coat, lethargy, hunched back, reluctance to move. Mice that survive two weeks are sacrificed. At termination, blood, liver and spleen samples are streaked onto Congo red agar plates, incubated at 28° C. for 2 days and analyzed for the presence of Y. pestis CFU. Brochial lavage samples are collected for determination of IgA responses. Total PBMCs are isolated from the spleen and used for immunological studies, such as determination of cytokine expression profiles after vaccination and T cell proliferation. Blood is obtained by heart puncture and antibody titers are measured by standard and isotype ELISA.

Determination of vaccine efficacy: At termination, serum, intestinal, nasal, vaginal and tracheobronchial washes, and spleen cells are collected from euthanized mice to assess the development of specific immune responses. The antibody responses are determined by ELISA analysis of serum for the presence of IgG, IgG1, IgG2a and IgG2b isotypes of antibodies specific to the purified, recombinant Y. pestis proteins and whole bacterial lysate antigens. The CMI responses are determined by in vitro culturing of spleen cells in the presence of purified Y. pestis F1, LcrV and YopD antigens and whole bacterial antigen and then IL-4 (Th2 cytokine) and IFNgamma (Th1 cytokine) secretion are quantified by antigen-capture ELISA with the culture supernatants. Furthermore, effector T cells are enumerated by ELISPOT. Secretions are assayed by ELISA for total corresponding Ig isotype concentrations in order to compensate for variations in Ig secretion and flow rate, or the variable dilution of secretions collected by washing. To verify whether specific IgA antibody-secreting cells (ASC) are generated at mucosal effector sites such as salivary glands, respiratory or intestinal lamina propria, these tissues are harvested from euthanized mice and processed to isolate mononuclear cells for ELISPOT assay (see details below).

We expect that mice immunized with the Lactobacillus vaccine candidates will develop a circulating IgG and mucosal IgA antibodies against the immunizing Y. pestis antigen, and particularly bias the response away from Th2 towards Th1 as measured by IgG subclass and CMI responses. From the published results with parenteral immunization with F1-LcrV chimeras, we expect that a combination of the Lactobacillus expressing the three antigens proposed will provide superior protection against challenge than either antigen alone.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above (including U.S. provisional application 60/812,595, filed Jun. 12, 2006) and in the figures, are hereby incorporated in their entirety by reference.

1. A Lactobacillus bacterium,
which expresses a recombinant polypeptide comprising a lipoprotein signal sequence from the OspA protein of Borrelia burgdorferi, or an active variant of the signal sequence, operably linked to one or more heterologous polypeptides of interest,
and/or
which comprises an expressible polynucleotide encoding a recombinant polypeptide, wherein the polynucleotide encodes a lipoprotein signal sequence from the OspA protein of Borrelia burgdorferi, or an active variant thereof, which is operably linked to one or more heterologous polypeptides of interest, and, optionally, wherein the sequence encoding the signal sequence is modified so that the codon usage is optimized for expression in Lactobacillus.

2. An immunogenic composition, comprising the bacterium of claim 1.

3. A vaccine, comprising the bacterium of claim 1 and, optionally, an adjuvant.

4. The bacterium of claim 1, wherein the Lactobacillus bacterium is L. plantarum.

5. The bacterium of claim 4, wherein the L. plantarum is L. plantarum strain 256.

6. (canceled)

7. The bacterium of claim 1, wherein the signal sequence is represented by SEQ ID NO:1.

8-10. (canceled)

11. The bacterium of claim 1, wherein the heterologous polypeptide(s) of interest is from a bacterial, viral, fungal or parasitic pathogen.

12. The bacterium of claim 1, wherein the heterologous polypeptide(s) of interest is from Yersinia pestis.

13-18. (canceled)

19. The bacterium of claim 1, wherein the heterologous polypeptide(s) of interest is a polypeptide from Borrelia burgdorferi other than the OspA protein.

20-21. (canceled)

22. The bacterium of claim 1, wherein the heterologous polypeptide expressed by the bacterium is more immunogenic than a form of the heterologous polypeptide lacking the lipoprotein signal sequence.

23. A pharmaceutical composition comprising a bacterium of claim 1 and a pharmaceutically acceptable carrier.

24. A method for eliciting an immune response to one or more polypeptides of interest in a vertebrate, comprising administering to the vertebrate an effective amount of a bacterium of claim 1 which expresses the polypeptide(s) of interest.

25. The method of claim 24, wherein the vertebrate is a human.

26. The method of claim 24, wherein the bacterium is administered orally or intranasally.

27-29. (canceled)

30. The method of claim 24, wherein the bacterium is live.

31-35. (canceled)

36. A method for inhibiting the infectivity and/or pathogenicity of a pathogen in a subject, comprising administering to the subject an effective amount of a bacterium, of claim 1, wherein the heterologous polypeptide(s) is derived from the pathogen, under conditions in which the bacterium is effective to elicit an immune response that inhibits the infectivity and/or pathogenicity of the pathogen.

37. A method for delivering an immunogenic polypeptide to a subject in need thereof, comprising administering to the subject an effective amount of a bacterium of claim 1, which expresses the polypeptide.

38. A method for enhancing an immune response in a host to a polypeptide of interest, comprising introducing into a Lactobacillus bacterium an expressible polynucleotide encoding a recombinant polypeptide which comprises a lipoprotein signal sequence from the OspA protein of Borrelia burgdorferi, or an active variant thereof, which is operably linked to the polypeptide of interest, and, optionally, wherein the polynucleotide encoding the signal sequence is modified so that the codon usage is optimized for expression in Lactobacillus,
wherein the presence of the signal sequence results in an enhancement of the immune response to the polypeptide of interest when the bacterium is introduced into the host.

39. A method for making a Lactobacillus bacterium which expresses a recombinant polypeptide comprising a lipoprotein signal sequence from the OspA protein of Borrelia burgdorferi, or an active variant thereof, operably linked to one or more polypeptides of interest, comprising
introducing into the bacterium an expressible polynucleotide encoding a recombinant polypeptide which comprises a lipoprotein signal sequence from the OspA protein of Borrelia burgdorferi, or an active variant thereof, which is operably linked to the polypeptide of interest, and, optionally, wherein the polynucleotide encoding the leader is modified so that the codon usage is optimized for expression in Lactobacillus.

40. A kit comprising a bacterium of claim 1 and means for packaging the bacterium.

41-46. (canceled)
